SEQUENCE LISTING (49 sequences)

SEQ ID NO: 1 - protein, 496 aa, Arabidopsis thaliana
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 2 - protein, 92 aa, Arabidopsis thaliana
ASGSSGRVNSGSKGFDFGSDDILCSYDDYTNQDSSNGPHSDPAIAASNSNKEFHKTRMARSSVFPTSSYSPPEDSLSQDITDTVERTMKMYA

SEQ ID NO: 3 - protein, 61 aa, Arabidopsis thaliana
DNMMRFLEGLSSRLSQLELYCYNLDKTIGEMRSELTHAHEDADVKLRSLDKHLQEVHRSVQ

SEQ ID NO: 4 - protein, 21 aa, Arabidopsis thaliana
ILRDKQELADTQKELAKLQLV

SEQ ID NO: 5 - protein, 262 aa, Arabidopsis thaliana
QKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAP

SEQ ID NO: 6 - protein, 59 aa, Arabidopsis thaliana
PVLRSKYGELIEKLVSMGFRGDHVMAVIQRMEESGQPIDFNTLLDRLSGQSSGGPPRGW

SEQ ID NO: 7 - protein, 404 aa, Artificial Sequence
  Description of Artificial Sequence Synthetic polypeptide
MDNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 8 - protein, 435 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYAILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 9 - protein, 475 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 10 - protein, 234 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVPVLRSKYGELIEKLVSMGFRGDHVMAVIQRMEESGQPIDFNTLLDRLSGQSSGGPPRGW

SEQ ID NO: 11 - protein, 437 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAP

SEQ ID NO: 12 - protein, 496 aa, Artificial Sequence
  Description of Artificial Sequence Synthetic polypeptide
MASGSSGRVNSGSKGSDSGSDDILCSSDDSTNQDSSNGPHSDPAIAASNSNKESHKTRMARSSVSPTSSSSPPEDSLSQDITDTVERTMKMSADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 13 - protein, 496 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQSYMPPPPTQLQNTPAPVPVSTPPSQLQAPPAQSQFMPPPPAPSHPSSAQTQSFPQSQQNWPPQPQARPQSSGGYPTSSPAPPGNQPPVESLPSSMQMQSPYSGPPQQSMQASGYGAAPPPQAPPQQTKMSSSPQTGDGYLPSGPPPPSGSANAMYEGGRMQSPPPQPQQQQQQAHYLQGPQGGGSSPQPHQAGGGNIGAPPVLRSKYGELIEKLVSMGFRGDHVMAVIQRMEESGQPIDFNTLLDRLSGQSSGGPPRGW

SEQ ID NO: 14 - protein, 496 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQSSMPPPPTQLQNTPAPVPVSTPPSQLQAPPAQSQFMPPPPAPSHPSSAQTQSFPQSQQNWPPQPQARPQSSGGSPTSSPAPPGNQPPVESLPSSMQMQSPSSGPPQQSMQASGSGAAPPPQAPPQQTKMSSSPQTGDGSLPSGPPPPSGSANAMSEGGRMQSPPPQPQQQQQQAHSLQGPQGGGSSPQPHQAGGGNIGAPPVLRSKYGELIEKLVSMGFRGDHVMAVIQRMEESGQPIDFNTLLDRLSGQSSGGPPRGW

SEQ ID NO: 15 - protein, 496 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESYSYSHSQHGEDRVATPVPEPKKYENTSDAHNQQLALALPHQIAPQPQVQPQPQPQQHQYYMPPPPTQLQNTPAPVPVYTPPSQLQAPPAQSQFMPPPPAPSHPSSAQTQSFPQYQQNWPPQPQARPQYSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 16 - protein, 496 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQFFMPPPPTQLQNTPAPVPVSTPPSQLQAPPAQSQFMPPPPAPSHPSSAQTQSFPQFQQNWPPQPQARPQSSGGFPTFSPAPPGNQPPVESLPSSMQMQSPFSGPPQQSMQAFGFGAAPPPQAPPQQTKMSFSPQTGDGFLPSGPPPPSGFANAMFEGGRMQFPPPQPQQQQQQAHFLQGPQGGGFSPQPHQAGGGNIGAPPVLRSKYGELIEKLVSMGFRGDHVMAVIQRMEESGQPIDFNTLLDRLSGQSSGGPPRGW

SEQ ID NO: 17 - protein, 496 aa, Artificial Sequence
  Description of Artificial Sequence Synthetic polypeptide
MASGSSGRVNSGSKGFDFGSDDILCSYDDYTNQDSSNGPHSDPAIAASNSNKEFHKTRMARSSVFPTSSYSPPEDSLSQDITDTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQWYMPPPPTQLQNTPAPVPVSTPPSQLQAPPAQSQFMPPPPAPSHPSSAQTQSFPQYQQNWPPQPQARPQSSGGYPTWSPAPPGNQPPVESLPSSMQMQSPYSGPPQQSMQAYGYGAAPPPQAPPQQTKMSWSPQTGDGYLPSGPPPPSGYANAMYEGGRMQWPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 18 - protein, 496 aa, Artificial Sequence
  Description of Artificial Sequence Synthetic polypeptide
MASGSSGRVNSGSKGFNFGSNNILCSYNNYTNQNSSNGPHSNPAIAASNSNKEFHKTRMARSSVFPTSSYSPPENSLSQNITNTVERTMKMYADNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 19 - protein, 496 aa, Artificial Sequence
  Description of Artificial Sequence Synthetic polypeptide
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQDNMMRFLEGLSSRLSQLELYCYNLDKTIGEMRSELTHAHEDADVKLRSLDKHLQEVHRSVQILRDKQELADTQKELAKLQLVQKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPPVLRSKYGELIEKLVSMGFRGDHVMAVIQRMEESGQPIDFNTLLDRLSGQSSGGPPRGW

SEQ ID NO: 20 - protein, 496 aa, Artificial Sequence
  Description of Artificial Sequence Synthetic polypeptide
QKESSSSSHSQHGEDRVATPVPEPKKSENTSDAHNQQLALALPHQIAPQPQVQPQPQPQQHQYYMPPPPTQLQNTPAPVPVSTPPSQLQAPPAQSQFMPPPPAPSHPSSAQTQSFPQYQQNWPPQPQARPQSSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAPDNMMRFLEGLSSRLSQLELYCYNLDKTIGEMRSELTHAHEDADVKLRSLDKHLQEVHRSVQILRDKQELADTQKELAKLQLVMASGSSGRVNSGSKGFDFGSDDILCSYDDYTNQDSSNGPHSDPAIAASNSNKEFHKTRMARSSVFPTSSYSPPEDSLSQDITDTVERTMKMYAPVLRSKYGELIEKLVSMGFRGDHVMAVIQRMEESGQPIDFNTLLDRLSGQSSGGPPRGW

SEQ ID NO: 21 - protein, 496 aa, Dunaliella salina
MDDMFEDLLAPPKKQPDPPPATTQQQQGTPEGGSSENGCVKQQQKEGGDGKDAEQQPPAPGLVGVSKEELQSLVSVAVEGAMDNLLGKFVKSLRLVLEDLGKRVDQQGTRLDSHSNEMKGALGEVLEQLESQAQNVHSRFTTVDMALKEVDRGVQALRDKQELMEAQATLARFSHTDAAPQQQQQQQQQKPGAGAPPAVKQEPAEPAPAAAAAPAAAPAPASSPSPAPAPAPTAAPASTPAVPLPQPFPTQAGLPHQYAAPGAAPPHMPPYHQQAPSQAAAALAPGAVPPHMLPPEPSAQYGGQPMQAYAGYNQPMPHASAVPPSSSPGPELAAAHSLPAYSQPMPAGYSQQPPTAPFPQPPQPMPMQPPQQFPPGAPYMPPTQPYGLHPSGSSGNLSMHAGPAPSPILGPRYPAPLSYPAPPVAPAAYRPGGGSVSQGPPSATRTSTRSVPVENIINDIAQMGFDRRQIMSVIADMQREGKAIDLNVVISRCLGS

SEQ ID NO: 22 - protein, 573 aa, Glycine max
MNTTPFMDKQIMDLTHGHGSSSSSTTQSQSKDFIDLMKEPPQHHHHHHLEDEDNDEEEKARGNGISKDDIVPSYDFQPIRPLAASNNFDSAAFSRPWNSDSNSNASPPVIKNYSSLDSMEPAKVIVEKDRSAFDATMLSEIDRTMKKHMENMLHVLEGVSARLTQLETRTHHLENSVDDLKVSVGNNHGSTDGKLRQLENILREVQSGVQTIKDKQDIVQAQLQLAKLQVSKTDQQSEMQTSAITNPVQQAASAPVQSQPQLPTPANLPQSIPVVPPPNAPPQPPPQQGLPPPVQLPNQFSQNQIPAAPQRDPYFPPPVQSQETPNQQYQMPLSQQPHAQPGAPPHQQYQQTPHPQYPQPAPHLPQQQPPSHPSMNPPQLQSSLGHHVEEPPYPPQNYPPNVRQPPSPSPTGPPPPPQQFYGTPTHAYEPSSSRSGSGYSSGYGTLSGPVEQYRYGPPQYAGTPALKPQQLPTASLAPSSGSGYPQLPTARVLPQAIPTASAVSGGSGSTGTGGRVSVDDVVDKVATMGFPRDHVRATVRKLTENGQSVDLNAVLDKLMNDGEVQPPRGWFGR

SEQ ID NO: 23 - protein, 538 aa, Selaginella sp.
MDNQGMGSHSEPFFDLLQPNTPSIAHASGSSSSNYVQNGPRRMDSSPTYSFNNDDVLPSYDFQPLRSNGSGGGARIEEAGGKFRQANPSFEQQVRDPPVTYEKYESTRSRHEFDKDAYDSATAAAVERTMKKYADNLLRVLEGMGGRLSQLEAATQRLEVAFEKSKSANANNHGETDGRLRMLENMLREVQRGVQVVRDKQEINEAQFQLKLQQDKTEAPTTKVEVQAPPVASSPQQPPPMPQPPQALDSSVHQQQAPPPPPPLPVVHQPPPPTHIQQSPHPPQHVPHAIQQQQQQPSYSYPPQNPAAPPPPPPPPMQQPHPQPYPHQPEAPPYPPAPVPVSHPQGPPHHSQAPPVNYSLDIPSYMPPPPPPQSYGAPPPPPPRQHQQQQQQQQHGPPPPQMYDSLPGRTGSGPLALPPPPSAYQQQSYETSGYGGGGVNYGRMHSGGGGGGGGGYPHLPTAQPIQQSLPSARPASRSGVDDVIDKVAAMGFPRDQVRATVQRLTENGQAVDMNVVLDKLMNGGGSDAGPPKAGWFGR

SEQ ID NO: 24 - protein, 640 aa, Unknown
  Description of Unknown Wollemia nobilis sequence
MEHQELGEGKENFLGFAPSGSSNPPSVNGNPSISRSGYKVTEGSAPGFDFSSEDILSSYEYNKKQNFSDGHYVAPSRLSNFPSDSYLNSSRSDRFRESRTAKPYANEQSQEDDNRYNEIVGTVERTMKKYADNLLKVLDGMSNRLMQLELVNERLERSVGEMRADMAEDHKENGERFRMLEDHVHEVHRTIQILRDKQEIAEAQTELAKLQLARKESSSNFQSPEDKTLTSSTLSEVKKEHAFQPQNVQAQLRSSNPAFPALPALPAPPQSSPSPSLPMPAREQCQSLLPQQQQPAQVSMVQQSPVTSFPLQQVAQLPQQPNVMLMQPYYPQQQGQIQPVPQAPQAGQVPHIQQQPPQPAVAAPPQVQNLPYGCQPQHIQNIPNQSSQHVQRPQIQQMPRLQSQPPPQTQMQPQPLSQQPHLPQQAQMRPNIYSGQTHGVPPEAFAYAPETGQHQTQAPYQGGPSSIPSEASMYNYGGPPQIIQPSSQGQVSIQSHRPQYPPSDSSNASSALVPPPVGHPMHGYSAYNSPPRPAPSPYGVPFSGAPQTTPFPGAYMRFPSAQQQYAHPSGNAVPNTSGGHLPSSHAFDDLVEQVATMGFSRDQVRVTIQQLTESGQPVDMNSVLDRLNNSPGPSQRGWYN

SEQ ID NO: 25 - protein, 552 aa, Theobroma cacao
MASGSSGRGNSGGSKGFDFGSDDILCSYEDYGNQESSNGSHAEPVVGTNSSAKDFHKGRAARSIFPPNAYSQPEDSFSTDVTATVEKTMKKYADNLMRFLEGISSRLSQLELYCYNLDKTIGEMRSDLVRDHVDADLKLKSIEKHLQEVHRSVQILRDKQELAETQKELAKLQLVQKESSSSSHSQSTEERASPPASDSKKTDHTSDMQSQQLALALPHQVAPPQQPVVPHSQASPQNLTQQSYYIPPNQLSNSQAQVQAPAPAPVPTPAPAPAPAPIQHPQSQYLPSDSQYRTPQIPDISRMPPQPTQSQVNQVPPVQSFPQYQQQWPQQLPQQVPQQQSSMQPQMRAPSTPAYPPYPPTQSTNPSLPEALPNSLPMQVPYSGVPQPVSSRADTIPYGYGLPGRTAPQQPQQIKGTFGAPPAEGYTAPGPHPPLPPGSAYMMYDSEGGRPLHPPQQPHFSQGGYSPANVSLQTPQTGTGPNVMIRNTSHSQFIRSHPYSDLIEKLVSMGFRVDHVASVIQRMEESGQPVDFNAVLDRLNVHSSGGSQRGGW

SEQ ID NO: 26 - protein, 634 aa, Marchantia polymorpha
MDSSLGIGTNHQPGAQNEPFFDLLQPAVTSSSSLGQNPPQNSSKMENSGEFNFSDDVLPSFDFQPIRTSGAPPLKTSNSGAGRMEESRSRQASPPPSYSSYEPMVRRSREPPPTYEAPLPRSQEHEKESFETATVAAVERTMKKYADNLLRVLEGMSGRLSQLESSTQRLEELYGEIRNDVVNNHGEVDGKLRSLENHILEVQRGVQLLRDRQELAEAQSQLAKLQAVTKSDVAPHNSAPSAPPPVIEQLPELSRASSGKALLEDSQQQMSNVASSHYQQPQPQHLQQLQLQLPSVPSHSLPQPLPQQQQQPQPQAQQHQPQQQQRNPSKKKGKGGVHQGPQMQQQSEVSHQILQQQQQQQQPPPPPPPPQQMSHSQHSPPPPPPPPPSQMTMPFYSQQQQPLPQAPPPMPTYGHQPEAPAYNQHPQGPHHVPPTPQSYPSDLPSYHPSNYGPPGSGLAQPPRQSSQIPPSSHIQQHHNVPMYDPSLARNGSGQLALPPPYLPQAQQVSNSPIYEPQSPGSGYPSSSYRVAQPVPSAPSGGGYPRLPVAQPLPHAMPAGGSGGGPPGTPPLSTNRVPIDEVIDKVTAMGFSKDQVRAVVRRLTENGQSVDLNIVLDKLMNGGDAQPPPKGWFGR

SEQ ID NO: 27 - protein, 553 aa, Chlamydomonas reinhardtii
MEDDLFGDLLGGPKPKPSNLTSPTGTASKDGHAGKAKTSAASANGADEEASGSGAATRSENAEKVTLSADDLAALVDKGVHAAMEATFSKFVRSLRTVLEDMTRRVSAQDVTLAELRHSVDELRDTVAAQPADLHIRFSNLDTAFKEVERNVQGIRDKLELQEAQALLAQMSSDVRAKGSSTSSAGAAPAAAAAPEAAAAPAAASAPAPAPAAAAPAAAPVAPAPAAAPAPAPVAQQAPVAPQAPMPAPVTQQAPAVGAPMPGMQYGAPQQQQAPQLQQQQQQQQQPQQQQQQLPPHMQPYGAPAPAPGMPGAPPLPMQPQQLQLQQQPSMEAKPVMQQPQQQQQQPQQQPYGAPGYPQYQQQPQQMPPPGVPDQGHYGAPAALPGPAPGGYPAGPYGGMPPQEAPRAPVMPQQHMAPPHMGVPPPAAAPRMDHPPPGAPPPPGMAYPAPPAMHAYPPPPAVPSYGRPQAAPPPTYRSPMPGPGPVSAPPGPPGGAPGGPPGTASRTVPLDQIIADIAQMGFSRGDVLNAVNNLQMSGKALDLNTIIDKLTRG

SEQ ID NO: 28 - protein, 565 aa, Klebsormidium sp.
METNKGGKYPAPSFSTENEPFYDLLKTGNNANQQSSLSGVATNPVDFGENILPSYDFHPTRPAPSLNNGNKMMSPTLSEQSLDGKSSTSEPLHGKQERSVADVDDSKDAVAAVERTMKKYADNLLRVLEDMRGKLTQLERTTDRLESTVAELQNRSADQHGELDGRVRGLEHVLREVQRGVQLLRDKSELQEAQAELAKMQMTTTAAKPPLPAQAPPALTAPPQTFPALTAPPLVPEEPAKPAAPMQMQPQPQVEQQPAPAPVPLPSAPSAPPQQLSVPVPQYQAPPKPPASPHPRHPPQPQQPQGPSGPAPRPRQYGPQAPPYMQRPPPQQQQEAPAYLPQGYGQQAGAPPHQMPPPPPQPQQGPPRQGYEGAPPQGAPHPGGRLALPPPPGSYGPPPPQGYSERPGSTGGYDRPPSASYDRPPTSGYERQAPPPFERPPPPNYDRQSGYEPRVPASPYGPPPQYGAGGPPPAPGTYPRLQMAQPVQSSEPPRTAGSGGPAQLSTSKMPIEQVIDDVAAMGFHKDEVRSIVRQLTETGKSVDLNIVLDTLMTRSGGAAPTGRSW

SEQ ID NO: 29 - protein, 403 aa, Unknown
  Description of Unknown Bathycoccus prasinos sequence
MEDDDPFDFKIGVEKNALNSGKKTTTEAMMKSMMMKPSSTTLESSSFTSFGEEEKKTMTMNDGVKGIPESKAPSSTKTDEDQKKKKNDDDDDAKVNATIESFSTETKVILTTLGKILERLEALELVALRNAKEVARVENALHGFIVGQARKENGKEPITSANLFAVVDSSEEEEEEEEEEEKIEEEIKENIVLRAGSGRSRRPPTPEGAHHPPHYPPHNPPPHHPPPPHAHHQHHQDPYGPPSFARGGRGGPPHPHPPPPPHERSGSPSGESAAHYPGIDHHLHPHPHRSPPPPHHGGPSSPPPHHGHPPPPSHVQHDPYGTYHPSPPPPPQVLASSYPSPPPPSPPQVQNEDIPLDVIVGEFASMGFTRDEVMTVLGKMEARNEQKEMNSILDKLMAGEGKL

SEQ ID NO: 30 - protein, 493 aa, Solanum lycopersicum
MDLSTNNDFINLHDDQHHITAGVNHPVRPIESFPNCSIHWAPDTKTNTNYSSPDSIEPAKLIVEKDLSTIDASLLSEIDHTVKKYADNLLHAIESVSARLSQLETRSRQIEDFVVKLKLSVDNNHGNTDGKLRLVENILREVQDGVQVIKNKQDIMETQLQLGKLQVPKEIDSSIVDSAHHRASAPLQSHQQFPPVVLAQPPSPLPPPNAPPPPLQQKIPSQVELQDQFPQNLIPSGTQRETYFPLTGQAPENSSQQNQQSAPHQRLQTSIPPPPHQQYLPFPSSLYTQPPVPSQAHSPLPSVNPSQSQPPLIHHPEERHFIASQTYPQANTSQFPSHPSSGAPVSHHFYAAPANLFEPPSSRQGSGFSSAYGPSTGPGESYPYSGSTVQYGSGSPFKSQQLASPLMGQSGGNGYPQLPTTRILPQALPTAFAVSSGSSSPRTGNRVPIDDVVDKVTNMGFPRDQVRATVQRLTENGQSVDLNVVLDKLMNGG

SEQ ID NO: 31 - protein, 539 aa, Coffea canephora
MASGSAGRPSNSGSKPFNFVSDDILCGPYEDYGNQDGSNGTSHSDPAIGATSAKEFHKNRMARSSVFPAASYSPPEESSFNQDVIATVERTMKKYADNLMRFLEGISSRLSQLELYCYNLDKSIAEMRSELGGDHTEAETKLKSLEKHLQEVHRSVQILRDKQELAEAQKELAKLHLAQKESSSASNLPQKEERVSAPASDAKKSENSSDSHGQQLALALPHQVPQPQQQQPPSVAPPPPMPSQSVPQAQAYYLPPHQLPNVPAAASQPSQGQYLPPDSHYRAPQLQDVSRVAPQPAQSQVNQAPQVQTIPSYQPQWPQQLPQQVQPLPQQSVQPQIRPSSPPVYSSYLPNQANPPPPEALPNSMPMQVPFSGISQPGPVRAETVPYGYGGAARPVQPQPQPQHLKATYASPADGYAASGPHPTLSPGNTYVMYDEAGRPHHPAQQPHFPQSPYPPTTMPPQNLQPNTGSNLVVRPPQFVRNHPYGDLIEKVVSMGYRGDHVVSAIQRLEESGQPVDFNAVLDRLNGHSAGGPQRGWSG

SEQ ID NO: 32 - protein, 547 aa, Arabidopsis thaliana
MQSFDLIKSALFSDKQIMDLMNDNSNNSQDGDHQNYRVGDNGLESKKEAIFPSYDFQPMRPNASAGLSHHALDLAGSVNSTAARVWDASDPKPVSASSARSYGSMDSLEPSKLFAEKDRNSPESAIISAIDRTMKAHADKLLHVMEGVSARLTQLETRTRDLENLVDDVKVSVGNSHGKTDGKLRQLENIMLEVQNGVQLLKDKQEIVEAQLQLSKLQLSKVNQQPETHSTHVEPTAQPPASLPQPPASAAAPPSLTQQGLPPQQFIQPPASQHGLSPPSLQLPQLPNQFSPQQEPYFPPSGQSQPPPTIQPPYQPPPPTQSLHQPPYQPPPQQPQYPQQPPPQLQHPSGYNPEEPPYPQQSYPPNPPRQPPSHPPPGSAPSQQYYNAPPTPPSMYDGPGGRSNSGFPSGYSPESYPYTGPPSQYGNTPSVKPTHQSGSGSGAYPQLPMARPLPQGLPMASAISSGGSGGGSDSPRSGNRAPVDDVIDKVVSMGFPRDQVRGTVRTLTENGQAVDLNVVLDKLMNGDRGAMMQQQQQQPPRGWFGGR

SEQ ID NO: 33 - protein, 511 aa, Arabidopsis thaliana
MNTCQFMDKQIMDLSSSSSLPSTDFIDLMNNHDGDDHQKKQVIGDNGLDSKKEVIVPSYDFHPIRPTTAARLSHSALDLAGSTTRVNWSASDYKPVSTTSPNTNFGSLDSIEPSKLVPDKGQNVFNTTIMSEIIDRTMKKHTDTLLHVMEGVSARLSQLETRTHNLENLVDDLKVSVDNSHGSTDGKMRQLKNILVEVQSGVQLLKDKQEILEAQLSKHQVSNQHAKTHSLHVDPTAQSPAPVPMQQFPLTSFPQPPSSTAAPSQPPSSQLPPQLPTQFSSQQEPYCPPPSHPQPPPSNPPPYQAPQTQTPHQPSYQSPPQQPQYPQQPPPSSGYNPEEQPPYQMQSYPPNPPRQQPPAGSTPSQQFYNPPQPQPSMYDGAGGRSNSGFPSGYLSEPYTYSGSPMSSAKPPHISSNGTGYPQLSNSRPLPHALPMVSAVSSGGGSSSPRSESRAPIDDVIDRVTTMGFPRDQVRATVRKLTENGQAVDLNVVLDKLMNEGGAPPGGFFGGR

SEQ ID NO: 34 - protein, 550 aa, Physcomitrella patens
MLVDQMEYQGQQGSGGPQDDAFYELLSSTALANAKKQQQQQHQFEQQNHQQQQQQQFDSRSEEGLPNYDFQSTSSSYGGVVANGEDMRKAPSVMPVVESSHPPHFPTYPPGSSYSNARQHLPVPSFVESSPPRQEKGNAEAATVAAVEQTMKKYADDLMRMMESMAGRIGQLESSTRRLEQIMTDFKGGSEKSQGVSGGKLLLIETMLSEVQRGVQELRNKQEVMDAQSTIGKLQLGDEGVSSSVHSQTSLEPPPAQSPRAPQMPETPPYPMGPLPHAPHHPPGHLPPYMVPPQLVGLAPPPPPPPAPEPHYQPSQQGPPPPPPPPPQQSYHSQQLQQQSTPPSAHPHGPFPQPPELPPYGATPQGPYKGQSGSFGQDAPPPSYGGRPHHMPQTGLGGSQMYDQSGGIPPYQSQGRPAAPAYDQPIGLPPPGYFNPGYRSGQQTPSAPSSGAGGYPRLPTAQPVQHAMPTAREREGAQPSSGATPLSTNRLSIDEVIDKVAVMGFSKDQVRAVVRRLTENGQSVDLNVVLDKLMNGDGGAQPPKGWFQRG

SEQ ID NO: 35 - protein, 548 aa, Solanum tuberosum
MASGSSGRPSNSSGSKGFDFGSDDILCSYEDYPHQDASNGTHSDPAIATNSAKEFHKNRMTRSSMFPTSTYSPPEESSFNQDMICTVEKTMKKYTDNLMRFLEGISSRLSQLELYCYNLDKSIGEMRSDLVRDHGEADLKLKALEKHVQEVHRSVQILRDKQELAETQKELAKLQFAQKEPASANNSQQNEDRNAQPVSDSNKGDNSTDVNGQELALALPHQVAPRAPLTNQPVEQPQQAPPQPIPSQSMTQSQGYYLPPVQMSNPPAPTHLSQGQYLSSDPQYRTSQMQDLSRLPPQPAAPPGNQTPQIQSMPQYQQQQWTQQVPQQIQASQQVQQHQLPTVQQQGRPSSPAVYPSYPPNQPNPSPEPVPNSMPMQMSYSAIPQSVACRPEAIPYGYDRSGRPLQSQPPTQHLKPSFGAPGDGYATSGPHPSLSAGNAYLMYDGEGPRGHPSQPPNFPQSGYPPSSFPPQNAQSSPSPNHMVRPPQLMRTHPYNELIEKLASMGYRGDHVVNVIQRLEESGQTVDFNTVLDRLNGHSSGGPQRGWSG

SEQ ID NO: 36 - protein, 498 aa, Solanum lycopersicum
MASGSSGRSNNAGSKGFDFASDDILCSYEDYANQDPSNGTHSDSVIAANSAKEFHKSRMTRSSMFPAPAYSPPEESSFNQDMICTIEKTMKKYTDNLMRFLEGISSRLSQLELYCYNLDKSIGEMRSDLVRDHGEADSKLKALEKHVQEVHRSVQILRDKQELAETQKELAKLQLAQKGSTSSSNSQQNEERSAQHLSDDKKSDDAPEVHGQQLALALPHQVAPQMANQQAPTQLSQGQFLSSDPQYRNPQMQVTPQRAAPQVNQTQQLQSMPQYQQQWAQQVPQQVQQSQIPNMQQQARPASPAVYPSYLHSQPNPTPETMPNSMPMQVPFSGVSQPVASRPESMPYGYDRSGRPLQQQPATPHLKPSFGAPGDGYAASGAHPTLSPGNAYVMYDGEGTRAHPPPQPNFQQSGYPPSSFPPQNQQPAPSPNLMVRPPQQVRNHPYNELIEKLVSMGYRGDHVVNVIQRLEESGQPVDFNAILDRMNGHSSGGPQRGW

SEQ ID NO: 37 - protein, 477 aa, Glycine max
MASGSSGRGNSASKGFDFASDDILCSYDDYANRDSTSNGNHTDPDFHKSRMARTSMFPTTAYNPPEDSLSQDVIATVEKSMKKYADNLMRFLEGISSRLSQLELYCYNLDKSIGEMKSDINRDHVEQDSRLKSLEKHVQEVHRSVQILRDKQELAETQKELAKLQLAQKESSSSSHSQSNEERSSPTTDPKKTDNASDANNQQLYLPSDQQYRTPQLVAPQPTPSQVTPSPPVQQFSHYQQPQQQQQPPQQQQQQWSQQVQPSQPPPMQSQVRPSSPNVYPPYQPNQATNPSPAETLPNSMAMQMPYSGVPPQGSNRADAIPYGYGGAGRTVPQQPPPQQMKSSFPAPPGEMYGPTGSLPALPPPSSAYMMYDGEGGRSHHPPQPPHFAQPGYPPTSASLQNPPQGHNLMVRNPNQSQFVRNHPYNELIEKLVSMGFRGDHVASVIQRMEESGQAVDFNSVLDRLSSVGPQRGGWSG

SEQ ID NO: 38 - protein, 560 aa, Sphagnum fallax
MDAFGGASSGMGSVQTGSQNDVFYDLLSNSTSALNGGGQQKKRDLVETRVSSPVVDFGNEEVQPPRYDVQPSYDFQPSASALGNSKITAFSSGNLSSSLRPPLTSEPTVHYEKEVIENATLVAVERTMKKYADNLLHVLEGISGRLTHLESTTQRLEHMVTEFKGGADENSSATDGKLRALGNMLSEVQRSVQVLRDRQELAEAHSQLAKLQLSVREGAPSAPVATQAPEPRPQSPPPPRHSDALPQQQGQSTSRHNPQLPTPPPHMLPQQPSPPLLPQQLQLQAPPAVQPEPQYQQQSPQPPPPHSMSFYSQPPPPPPPPPPPQQQQGPPPSLQQQYSHPPEAPPYGTHPQGPHQGPPPPSANYADLPPQFMPFGNRPFPQQQPPPMQTLQPQAGSGGPPMYDTQAGGSSSSSMGLPPPYHSQGRPAVPNYDQQQMNAPAGYGSPAYHRMPQPAVPSAPSSGNGGYPRLPTAQPVQHALPTATATGPGPSGPAPLSTNRVPIDEIIEKVSSMGFSKDQVRAVVRRLTENGQSVDLNIVLDKLMNGGADVQPQKGWFGRG

SEQ ID NO: 39 - protein, 541 aa, Theobroma cacao
MNTSQFMDKQIMDLTSSSSSPPHNTNKDFIDLMNNPQNEDNHNQGSGISNKEGIFPSYDFQPIRPVSTSLDAAAVNNNPRSWSSGDSKTKNYGSLDSVEPAKVILEKDRNAFDTSIVAEIDRTMKKHTDNLIHMLEVVSARLTQLESRTRNLENSVDDLKVSVGNNHGSTEGKMRQLENILNEVQTGVHVLKEKQEIMEAQLHLAKLQVTKGDHPSETQNTVHVDTVQQAASAPFQSHQQLPPAASFPQSLPSVPPPPTVPPLVLPQQNLPPPVQHPNQFPQSQVPSVPQRDAYYPPPGHTQEAPGQQFPVPPTQQPQLPPAAPPHQPYQPVPPPQYSQPPQPVQLQPSLGHHPEEAPYVPSQNYPPNLRQPPSQPPSGPPSSQQYYGAPPQMHEPPSSRPGSGFSAGYIPQSGQSEPYAYGGSPSQYGSGSPMKMQQLPSSPMGQSGGSGYPQLPTARILPHALPTASGVGGGSGPSGPGNRVPVDDVIDKVTSMGFPRDHVRATVRKLTENGQSVDLNVVLDKLMNDSEVQPPRGWFGR

SEQ ID NO: 40 - protein, 331 aa, Unknown
  Description of Unknown Ostreococcus tauri sequence
MPSAREDIDPFDLLSPIASDARRRARAVTDEKTTATTTTGTMTNESRSIRHADADADAVRDEAMEKLISRVEALERVSRDGFARVGEVLERLTGRVETLSARVAAMRRDEEYDDEDSSDSSGDEAEEASEDVREEDGYADVPRRRGSPPRRRRRSPPRHHRGPPPPRRRGSPPPRHHRGSPPHHQHGPPPDHGGPPPHHHHGPPPLDHRGPPPHHHGPPPPHHHGPPPHQHGPPPPPSYEQMVPPTAYPSSPYPMYAPPPEPPRAPPPESPRSMAPPPVTSGAVPLEQMIGDFANMGFTRQQVMNAVSEMASSGQKIEVNSVLDRLMRAHA

SEQ ID NO: 41 - protein, 249 aa, Unknown
  Description of Unknown Wollemia nobilis sequence
MQQGPPNAMQISAYSQNPQPQQPSGQSVSIPFSQPEPTPSLAQHMPHSQMPTPALPGNYGPEPPYMPSNYGGSSSHQPPRSMPPPQLPASQRFSGSQQGYEPTFGRTSSGPLPFPPTYGPGLSGPPPYGDSQTYSGPSFRLPQKDSNPSGGGSSAGHPRLPTAKPLQHSLPVASSVNSSPSGSTSSSNRVPVDDVVDKVSSMGFPRDQVKMVVQKLTENGQSVDLNVVLDKLMNGGGGEIQPQKGWFGR

SEQ ID NO: 42 - DNA, 19 bp, Artificial Sequence
  Description of Artificial Sequence Synthetic oligonucleotide
ttacagcccccagactggc

SEQ ID NO: 43 - protein, 93 aa, Arabidopsis thaliana
MASGSSGRVNSGSKGFDFGSDDILCSYDDYTNQDSSNGPHSDPAIAASNSNKEFHKTRMARSSVFPTSSYSPPEDSLSQDITDTVERTMKMYA

SEQ ID NO: 44 - protein, 93 aa, Artificial Sequence
  Description of Artificial Sequence Synthetic polypeptide
MASGSSGRVNSGSKGSDSGSDDILCSSDDSTNQDSSNGPHSDPAIAASNSNKESHKTRMARSSVSPTSSSSPPEDSLSQDITDTVERTMKMSA

SEQ ID NO: 45 - protein, 262 aa, Artificial Sequence
  Description of Artificial Sequence Synthetic polypeptide
QKESSSSSHSQHGEDRVATPVPEPKKSENTSDAHNQQLALALPHQIAPQPQVQPQPQPQQHQSYMPPPPTQLQNTPAPVPVSTPPSQLQAPPAQSQFMPPPPAPSHPSSAQTQSFPQSQQNWPPQPQARPQSSGGYPTSSPAPPGNQPPVESLPSSMQMQSPYSGPPQQSMQASGYGAAPPPQAPPQQTKMSSSPQTGDGYLPSGPPPPSGSANAMYEGGRMQSPPPQPQQQQQQAHYLQGPQGGGSSPQPHQAGGGNIGAP

SEQ ID NO: 46 - protein, 262 aa, Artificial Sequence
  Description of Artificial Sequence Synthetic polypeptide
QKESSSSSHSQHGEDRVATPVPEPKKSENTSDAHNQQLALALPHQIAPQPQVQPQPQPQQHQSSMPPPPTQLQNTPAPVPVSTPPSQLQAPPAQSQFMPPPPAPSHPSSAQTQSFPQSQQNWPPQPQARPQSSGGSPTSSPAPPGNQPPVESLPSSMQMQSPSSGPPQQSMQASGSGAAPPPQAPPQQTKMSSSPQTGDGSLPSGPPPPSGSANAMSEGGRMQSPPPQPQQQQQQAHSLQGPQGGGSSPQPHQAGGGNIGAP

SEQ ID NO: 47 - protein, 262 aa, Artificial Sequence
  Description of Artificial Sequence Synthetic polypeptide
QKESYSYSHSQHGEDRVATPVPEPKKYENTSDAHNQQLALALPHQIAPQPQVQPQPQPQQHQYYMPPPPTQLQNTPAPVPVYTPPSQLQAPPAQSQFMPPPPAPSHPSSAQTQSFPQYQQNWPPQPQARPQYSGGYPTYSPAPPGNQPPVESLPSSMQMQSPYSGPPQQSMQAYGYGAAPPPQAPPQQTKMSYSPQTGDGYLPSGPPPPSGYANAMYEGGRMQYPPPQPQQQQQQAHYLQGPQGGGYSPQPHQAGGGNIGAP

SEQ ID NO: 48 - protein, 262 aa, Artificial Sequence
  Description of Artificial Sequence Synthetic polypeptide
QKESSSSSHSQHGEDRVATPVPEPKKSENTSDAHNQQLALALPHQIAPQPQVQPQPQPQQHQFFMPPPPTQLQNTPAPVPVSTPPSQLQAPPAQSQFMPPPPAPSHPSSAQTQSFPQFQQNWPPQPQARPQSSGGFPTFSPAPPGNQPPVESLPSSMQMQSPFSGPPQQSMQAFGFGAAPPPQAPPQQTKMSFSPQTGDGFLPSGPPPPSGFANAMFEGGRMQFPPPQPQQQQQQAHFLQGPQGGGFSPQPHQAGGGNIGAP

SEQ ID NO: 49 - protein, 262 aa, Artificial Sequence
  Description of Artificial Sequence Synthetic polypeptide
QKESSSSSHSQHGEDRVATPVPEPKKSENTSDAHNQQLALALPHQIAPQPQVQPQPQPQQHQWYMPPPPTQLQNTPAPVPVSTPPSQLQAPPAQSQFMPPPPAPSHPSSAQTQSFPQYQQNWPPQPQARPQSSGGYPTWSPAPPGNQPPVESLPSSMQMQSPYSGPPQQSMQAYGYGAAPPPQAPPQQTKMSWSPQTGDGYLPSGPPPPSGYANAMYEGGRMQWPPPQPQQQQQQAHYLQGPQGGGYSPQPHQAGGGNIGAP

(19) United States
(12) Patent Application Publication
(10) Pub. No.: US 2023/0416772 A1
(43) Pub. Date: Dec. 28, 2023

(54) FLOE1-MEDIATED MODULATION OF SEED LONGEVITY AND GERMINATION RATES

(71) Applicants: The Board of Trustees of the Leland Stanford Junior University, Stanford, CA (US); Carnegie Institution of Washington, Washington, DC (US)

(72) Inventors: Yanniv Dorone, Stanford, CA (US); Steven Boeynaems, Stanford, CA (US); Aaron D. Gitler, Stanford, CA (US); Seung Yon Rhee, Stanford, CA (US)

(73) Assignees: The Board of Trustees of the Leland Stanford Junior University, Stanford, CA (US); Carnegie Institution of Washington, Washington, DC (US)

(21) Appl. No.: 18/040,638

(22) PCT Filed: Aug. 6, 2021
(86) PCT No.: PCT/US2021/045103

§ 371 Date: Feb. 3, 2023

Related U.S. Application Data
(60) Provisional application No. 63/063,009, filed on Aug. 7, 2020.

(51) Int. Cl. C12N 15/82; C07K 14/415

(52) U.S. Cl. CPC C12N 15/8267; C07K 14/415

(57) ABSTRACT

Described herein are methods of modulating seed germination and seed longevity in plants by modifying FLOE1 level or activity; and plants generated by such methods.

### CROSS-REFERENCE TO RELATED APPLICATIONS

This application claims benefit of U.S. provisional application No. 63/063,009, filed Aug. 7, 2020, which is herein incorporated by referenced for all purposes.

### STATEMENT AS TO RIGHTS TO INVENTIONS MADE UNDER FEDERALLY SPONSORED RESEARCH AND DEVELOPMENT

This invention was made with Government support under contract DE-SC0018277 awarded by the Department of Energy, under contract DE-SC0008769 awarded by the Department of Energy, under contract 617020 awarded by the National Science Foundation and under contract NS097263 awarded by the National Institutes of Health. The Government has certain rights in the invention.

### BACKGROUND

Plant seeds are specialized propagation vectors that can mature to a quiescent desiccated state, allowing them to remain viable in harsh conditions anywhere from a few years to millennia (1, 2). Water is essential for life but plant embryos can survive extreme desiccation by accumulating protective molecules and profoundly changing their cellular biophysical properties (3, 4). Upon the uptake of water, called imbibition, seeds rapidly undergo a cascade of biochemical events and the resumption of cellular activities (5). Seeds can endure multiple hydration-dehydration cycles while remaining viable and desiccation tolerant (6). But once committed to germination, they are no longer able to revert to their stress tolerant state (5). Thus, poor timing of germination can severely limit the chances of seedling survival (7), especially in times of drought. Despite the fundamental importance of germination control for plant biology and agriculture, the molecular underpinnings controlling this decision remain incompletely understood.

### BRIEF SUMMARY OF ASPECTS OF THE DISCLOSURE

We identified an uncharacterized Arabidopsis prion-like protein. FLOE1, that phase separates upon hydration and allows the embryo to sense water stress. We demonstrated that the emergent properties of FLOE1 condensates are intimately linked to its biological function in vivo, where it functions as a negative regulator of seed germination in unfavorable environmental conditions. These findings provide evidence of a functional role of phase separation in a multicellular organism and have direct implications for plant ecology and agriculture, especially for generating drought resistant crops, in the face of climate change. Additionally provided herein are methods of modulating seed germination by modulating FLOE1 expression.

### BRIEF DESCRIPTION OF THE DRAWINGS

FIG. 1A-1L: FLOE1 is an uncharacterized seed protein that undergoes biomolecular condensation in a hydration-dependent manner. (A) Identification of genes enriched in dry Arabidopsis seeds. (B-C) The seed proteome is enriched for specific amino acids (B) and intrinsic disorder (C). Mann-Whitney. (D) The seed proteome is enriched for prion-like proteins. Binomial test. AT4G28300 is an uncharacterized prion-like protein, which we name here FLOE1. (E) FLOE1-GFP is expressed during embryonic development and forms condensates. (F) FLOE1-GFP forms condensates in embryos dissected from dry seed in a hydration-dependent and reversible manner. Cotyledons are shown. PSV denotes highly autofluorescent protein storage vacuoles in the dry state (see also FIG. S3C). (G) Cell to cell variation in subcellular FLOE1-GFP heterogeneity in response to solution salt concentration. Radicles are shown. * denotes nuclear localization. (H) Quantification of cellular FLOE1 heterogeneity as a function of salt concentration. Black line denotes the 95 percentile of 2M heterogeneity distribution. (I) Quantification of the percentage of cells per radicle that show FLOE1 condensation as a function of salt concentration. Four-parameter dose-response fit. (J) Quantification of the percentage of cells per radicle that show FLOE1 nuclear localization as a function of salt concentration. Gaussian fit. (K) FLOE1-GFP condensation is reversible by high salt treatment. Radicles are shown. (L) Scheme highlighting different FLOE1 behaviors upon imbibition.

FIG. 2A-2P: Molecular dissection of FLOE1 phase separation. (A) FLOE1 domain structure. CC=predicted coiled coil, DUF=DUF1421. Balloon plots show amino acid composition of the disordered domains. (B) Expression of wildtype FLOE1 in the human U2OS cell line (C-D) Expression of FLOE1 domain deletion mutants in tobacco leaves (C) and human U2OS cells (D). V=vacuole, C=cytoplasm, N=nuclear localization. (E) Summary of FLOE1 behavior in tobacco leaves and human cells. (F) Chimeric proteins containing both the FLOE1 nucleation domain and PrLDs from FLOE1 (QPS) or the human FUS protein form cytoplasmic condensates. Percentages display number of cells lacking or containing condensates. Average of 3 experiments. Arrowheads point at cytoplasmic condensates. (G) The number of QPS tyrosine residues alters FLOE1 phase separation in human cells and tobacco leaves. (H) FLOE1 phase diagram as a function of concentration and number of QPS tyrosines. (I) Number of QPS tyrosines affects intracondensate FLOE1 dynamics. Mobile fraction as assayed by FRAP is shown. One-way ANOVA. Purple band denotes WT mean+−SD. (J-K) QPS tyrosine-phenylalanine and tyrosine-tryptophan substitutions alter condensate morphology (J) and intracondensate dynamics compared to WT (K). One-way ANOVA. (L) DS deletion or DS tyrosinelphenylalanine-serine substitution alters condensate morphology. (M) TEM shows that mutant DS FLOE1 condensates have filamentous substructure that is absent in the WT. U2OS cells. (N) DS tyrosine/phenylalanine-serine substitution alters intracondensate dynamics. Student's t-test. Purple band denotes WT mean+−SD. (N) DS tyrosine/phenylalanine-serine substitution alters condensate morphology. Mann-Whitney. (P) Scheme summarizing synergistic and opposing roles of FLOE1 domains on the material property spectrum. * p-value<0.05, ** p-value <0.01, *** p-value<0.001, **** p-value<0.0001.

FIG. 3A-K: FLOE1 condensate material properties regulate its role in seed germination under salt stress. (A) floe1-1 seeds show higher germination rates under salt stress. Two-way ANOVA. Four-parameter dose-response fit. (B) Seedlings show developmental defects under salt stress. Three-week-old floe1 seedlings are shown are shown (see also FIG. S6G). (C) Seeds retain full germination potential under standard conditions after a 15-day salt stress treatment. (D) FLOE1 condensates are largely absent in ungerminated seeds after 15 days of incubation under salt stress. FLOE1 condensates appear within two hours after transfer to standard conditions (MS medium). (E) Scheme highlighting position of tested FLOE1 mutants on the material properties spectrum. (F) Representative images of mutant FLOE1 complemented lines upon dissection in water. Radicles are shown. (G) Close up pictures of WT and mutant FLOE1 condensates. Radicles are shown. (H) Quantification of FLOE1 condensate size. One-way ANOVA. (I) ΔDS FLOE1 condensates are not dependent on hydration. Radicles are shown. (J) Germination rate of WT, floe1-1 and complemented lines. One-way ANOVA. (K) Scheme highlighting role of FLOE1 in regulating germination and the effect of mutants with altered material properties. p-value<0.05, ** p-value<0.01, *** p-value<0.001, **** p-value<0.0001.

FIG. 4A-4H. Natural sequence variation tunes FLOE phase separation. (A) Arabidopsis has long and short FLOE1 isoforms. FLOE1.2 has larger condensates than FLOE1.1 in tobacco leaves. Mann-Whitney. (C) FLOE1.2 condensates recruit FLOE1.1, (D) FLOE1 has two Arabiclopsis paralogs that form larger condensates in tobacco leaves. Mann-Whitney. (E) Species tree of the plant kingdom with example species and their number of FLOE homologs. (F) Gene tree of FLOE homologs. Numbers highlight Arabidopsis FLOE1, FLOE2 and FLOE3 homologs. (G) Distribution of DS and QPS length differences between the FLOE1-like and FLOE2-like Glade among monocots and dicots. Mann-Whitney. (H) Examples of FLOE homologs from across the plant kingdom. N denotes nuclear localization. For full species names for (E,F):
Bpr-FLOE2L: homolog from Bathycoccus prasinos;
Ota-FLOE2L: hornolog from Ostreoccocus tauri;
Cre-FLOE2L: homolog from Chlamydomonas reinhardtii
Kni-FLOE2L: homolog from Klebsormidium nitens
Mpo-FLOE2: homolog from Marchantia polymorpha
Smo-FLOE2L: homolog from Selaginella moellendorffii
Wno-FLOE1L: homolog from Wollemia noblis #1
Wno-FLOE2L: homolog from Wollemia noblis #2
Gma-FLOE1L: homolog from Glycine max #1
Gma-FLOE2L: homolog from Glycine max #2
Stu-FLOE1L: homolog from Solanum tuberosum
Sly-FLOE1L: homolog from Solanum lycopersicum #1
Sly-FLOE2L: homolog from Solanum lycopersicum #2
Tea-FLOE1L: homolog from Theobroma cacao #1
Tea-FLOE2L: homolog from Theobroma cacao #2

FIG. 5: Amino acid composition of the Arabidopsis seed proteome. Average amino acid fractions are shown for seed-enriched proteins (Z>3) and the remainder of the proteome (Z<3), Mann-Whitney. * p-value<0.05, ** p-value<0.01, *** p-value<0.001, **** p-value<0.0001.

FIG. 6A-6C: FLOE1 and FLOE1 expression in Arabidopsis. (A) Tissue-specific expression of FLOE1 derived from ePlant (haps://bar.utoronto.ca/eplantl). (B) RT-qPCR analysis of different developmental stages shows peak expression in mature dry seeds, and a decrease in expression upon imbibition. “Dark”, “green” and “yellow” refer to the maturation stages of the siliques (from younger to older), which roughly correspond to 4-7, 8-10 and 11-13 days post-anthesis, and “imbibed” corresponds to seeds that were imbibed in sterile double-distilled water for 24 h. Col-0 (WT) plants were used. One-way ANOVA. **** p-value<0.0001. Mean±SD shown. (C) Expression of FLOE1 in developing embryos detected by GUS staining in FLOE1p:FLOE1-GUS transgenic

FIG. 7A-7G: FLOE1 forms condensates dependent on water potential. (A) YFP-FLAG localizes diffusely with modest nuclear enrichment in Arabiclopsis torpedo stage embryos without any granules or condensates forming. (B) GFP localizes diffusely with modest nuclear enrichment in imbibed dry seed-derived embryo radicles without any granules or condensates forming. (C) Autofluorescence of protein storage vacuoles in non-transenic control plants is dependent on hydration state. (D) Dissection in glycerin does not alter presence of FLOE1-GFP condensates throughout embryonic development (pre-desiccation). (E) Cycloheximide treatment does not prevent FLOE1-UP condensate formation in imbibed embryo radicles. (F-G) Incubation of FLOE1-GFP embryos in osmolyte solutions prevents FLOE1 condensate formation. Mannitol: Mann-Whitney. Sorbitol: One-way ANOVA, **** p-value 0.0001.

FIG. 8: Expression in tobacco leaves. Both N- and C-terminal GFP fusions condense into cytoplasmic condensates. V denotes vacuole, C denotes cytoplasm.

FIG. 9A-9B: Amino acid substitution mutants. (A) Domain architecture of FLOE1 with repetitively spaced aromatic residues highlighted. (B) Sequences of amino acid substitution mutants.

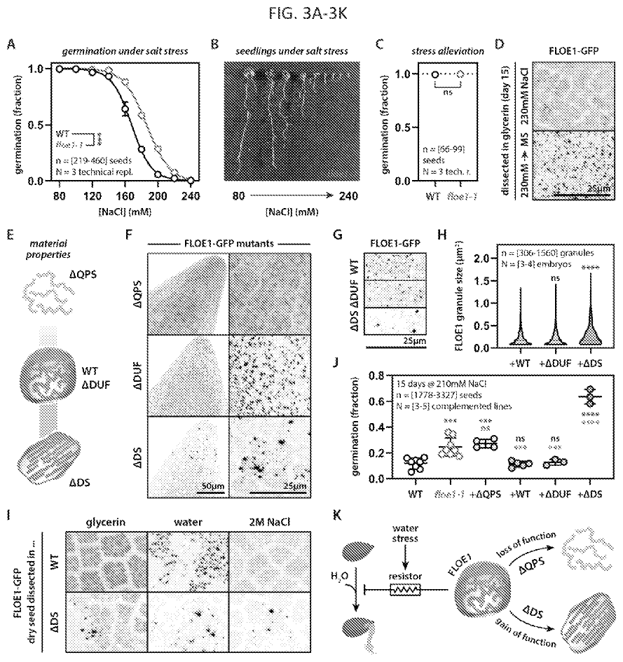

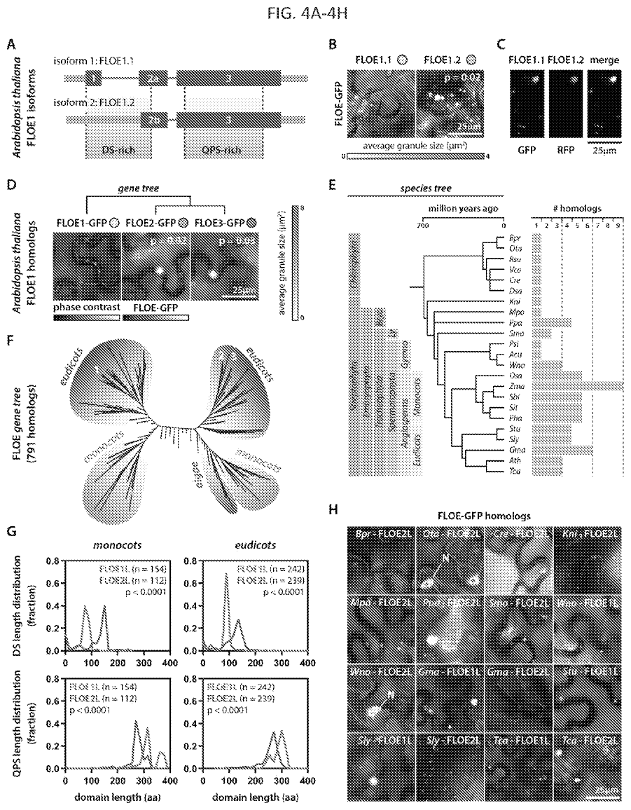

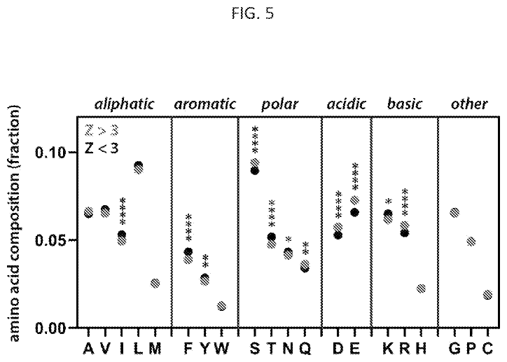

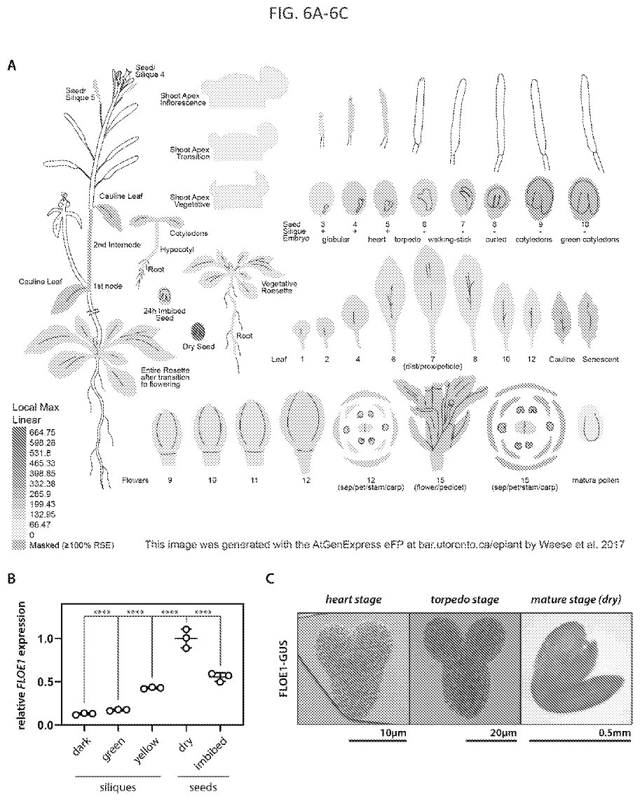

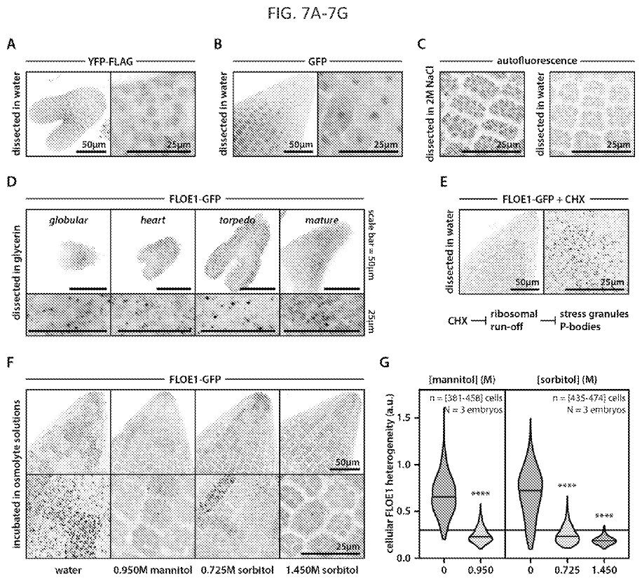

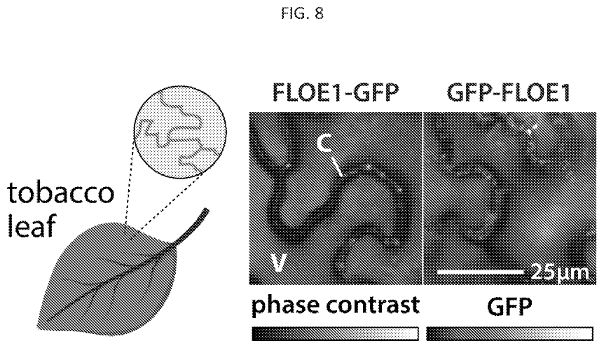

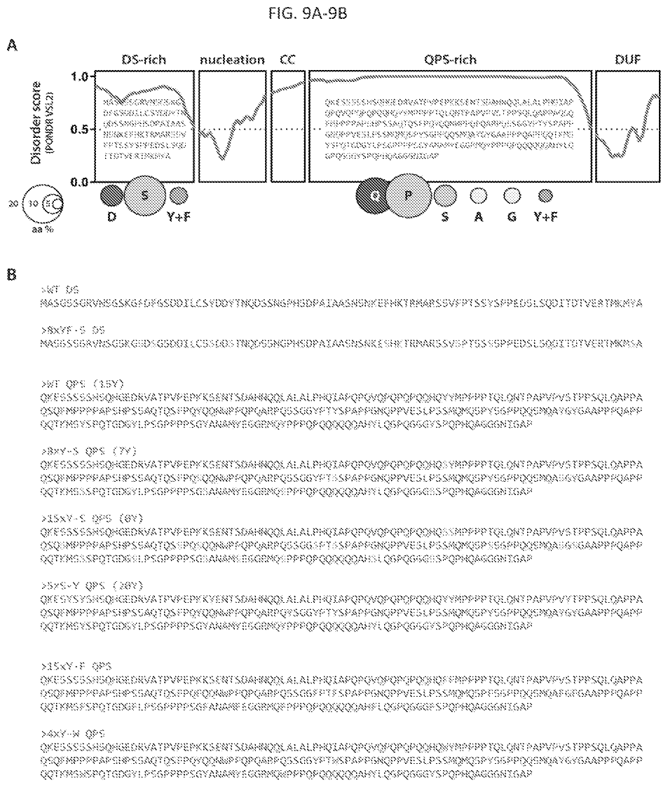

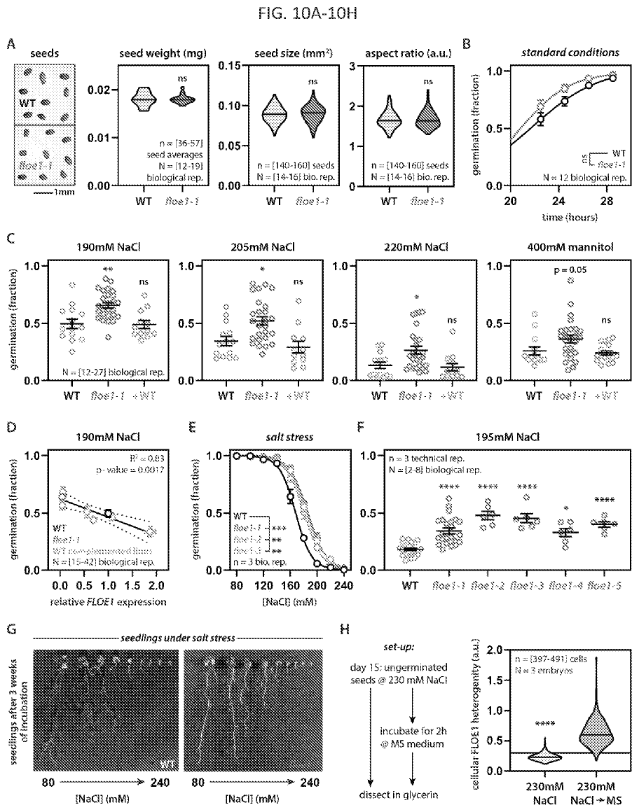

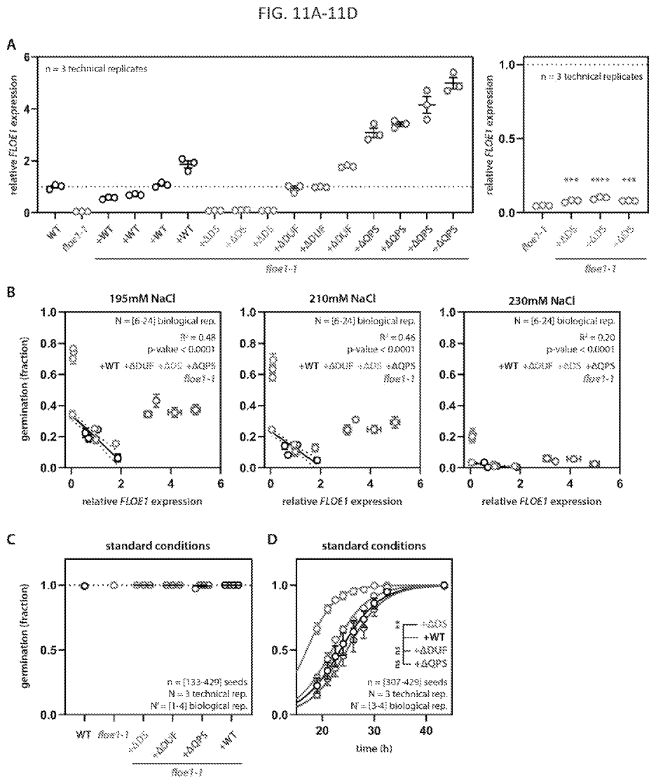

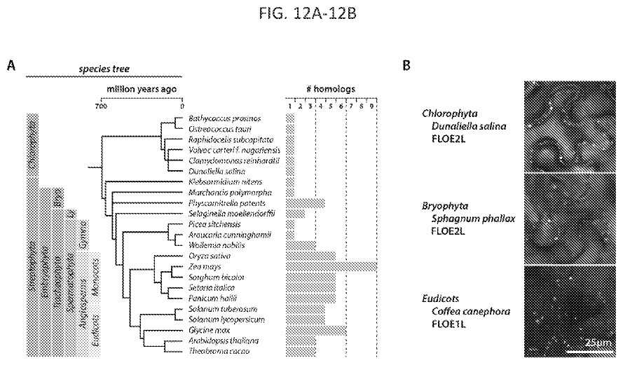

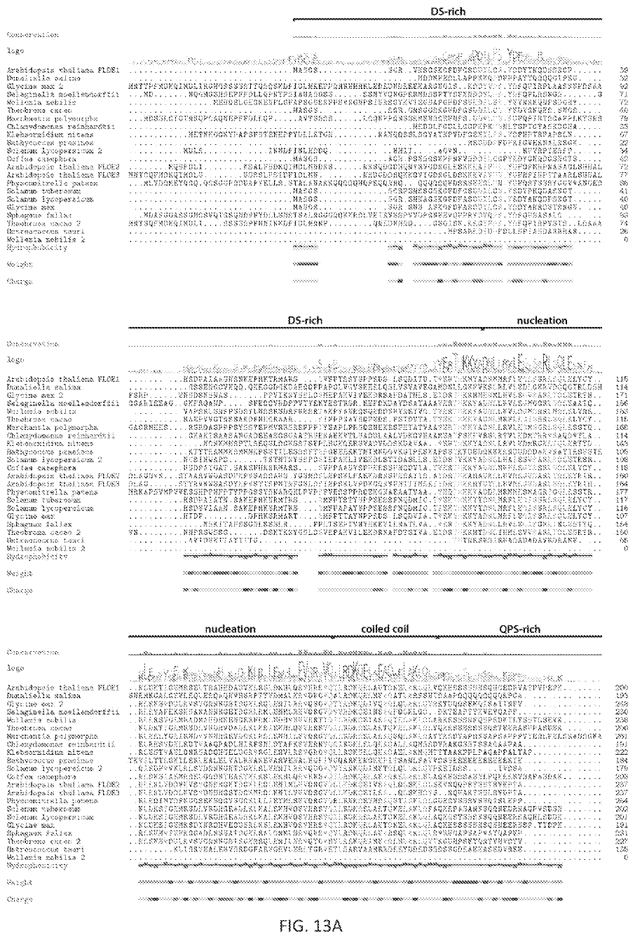

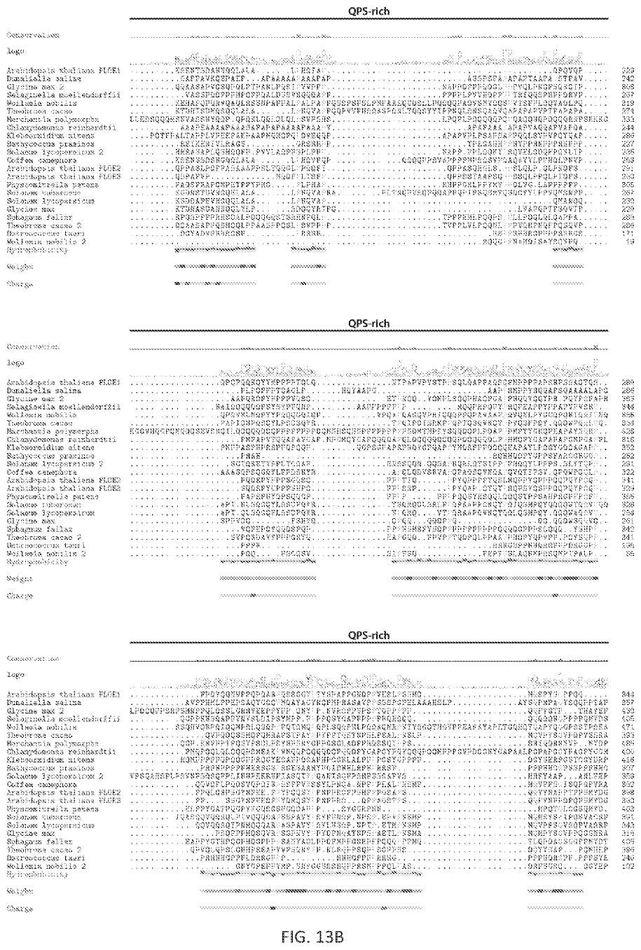

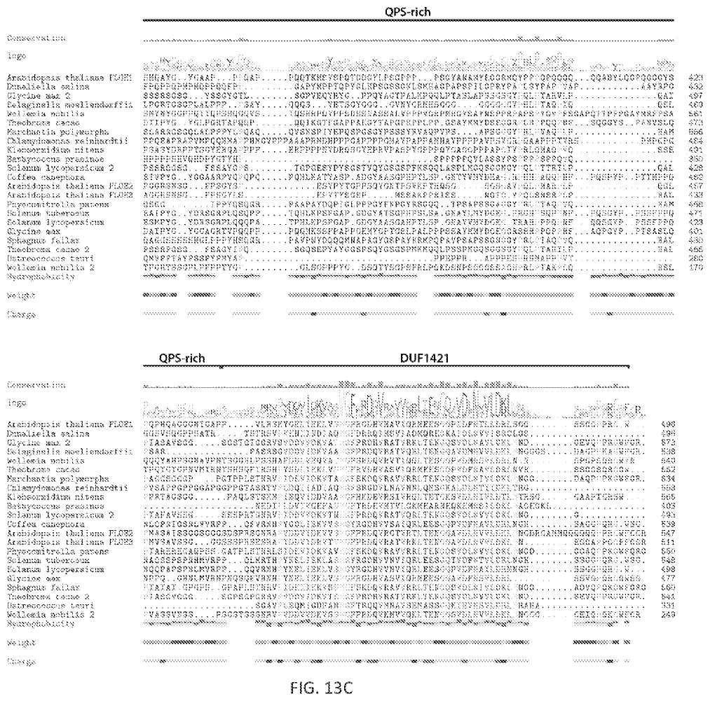

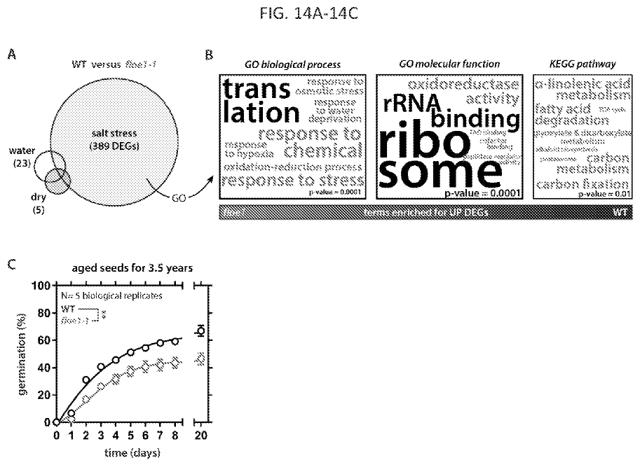

FIG. 10A-10H: FLOE1 function modifies germination rate under water stress, (A) FLOE1 deletion does not affect seed characteristics. Mann-Whitney. (B) FLOE1 deletion does not affect germination under normal conditions. Mean+−SEM. Four-parameter dose-response fit. (C) Increased germination of floe1-1 T-DNA line under water stress is rescued by WT FLOE1 complementation. Mean+−SEM. One-way ANOVA. (I)) Different FLOE1 WT complemented lines with different expression levels, as assayed by qPCR, show dose-dependent effect of FLOE1 function on germination under salt stress. Mean+−SEM. Linear regression. (E) Two CRISPR-Cas9 FLOE1 mutant lines show enhanced germination under varying salt stress conditions. Mean+−SEM. Four-parameter dose-response fit. Two-way ANOVA. (F) Four CRISPR-Cas9 FLOE1 mutants lines show enhanced germination under salt stress. Mean+−SEM. One-way ANOVA. (G) Both WT and floe1-1 seedlings show developmental defects upon germination under salt stress. floe1-1 picture is the same as in FIG. 3B and is shown for comparison. (H) Quantification of FLOE1 condensate formation upon alleviation from salt stress. Mann-Whitney, * p-value<0.05, ** p-value<0.01, *** p-value<0.001, **** p-value<0.0001.

FIG. 11A-11D: Mutant phenotypes are not due to differences in expression level. (A-B) Since FLOE1 is a dosage-dependent regulator of seed germination under water stress, we wanted to rule out that expression differences in the mutant lines would be responsible for the observed differences in their germination rates. We assayed FLOE1 expression levels in dry seeds via RT-qPCR (A). As shown before, there was a linear correlation between FLOE1 expression level and the germination rate (B). floe1-1 lines complemented with the ΔDUF mutant followed a similar trend, confirming that the DUF domain deletion does not affect germination in our assays (B). floe1-1 lines complemented with the ΔDS mutant showed low levels of transgene expression according to RT-qPCR (A, Right Panel. One-way ANOVA. *** p-value<0.001. Mean±SEM.) which was consistent with the sparser localization of the protein in radicles (FIG. 4F). Yet, despite these low expression levels, the ΔDS complemented lines consistently induced extreme germination rates, which we never observed for floe1-1 or WT complemented lines. floe1-1 lines complemented with the ΔQPS mutant showed high levels of transene expression according to RT-qPCR (B). Despite these high transgene levels, and robust protein expression in radicles (FIG. 4F), ΔQPS complemented lines had. germination rates similar to the parental floes-1 line, in stark contrast with WT complemented lines with higher relative expression, supporting the loss-of-function phenotype of this mutant. B: Mean±SEM. Germination data are representative of three independent experiments. (C) All complemented lines are able to fully germinate under standard conditions (43.5 h time point shown) Mean±SEM. Representative of two independent experiments. (D) ΔDUF and ΔQPS complemented lines have similar germination rates as WT complemented lines. In contrast, ΔDS complemented lines show faster germination rates under standard conditions. Mean±SEM. Two-way ANOVA. Average of 3-4 independent transgenic lines.

FIG. 12A-12B: Additional information on FLOE homologs. (A) Species tree as in FIG. 4E with full species names. (B) Additional examples of FLOE homologs that condense upon expression in tobacco leaves.

FIG. 13: Protein sequence alignimnt of tested FLOE homologs. Homologs from across the plant kingdom show extensive sequence variation in both the DS and QPS disordered domains but high conservation in the other domains. The sequence shown in the alignment are those that were tested in tobacco transient assays (see, e.g., FIG. 4 in which homologs were fused to GFP expressed in tobacco cells to determine where they localized to. What the tobacco transient assays show is that the FLOE homologs from the different species all form condensates that are either small like those of FLOE1 or much larger like those created by the ΔDS (DS deletion) FLOE1 version. The only exceptions are those that say “Ota-FLOE2L” and “Wno-FLOE2L”: these are particularly truncated homologs and they localize to the nucleus.

FIG. 14A-14C: RNA seq analysis of WT and floe1 seeds. (A) Venn diagram showing differentially expressed genes (DEGs) between wildtype and floe1-1 seeds under different conditions: dry seed (dry), normal imbibition (water), imbibition in 220 mM NaCl (salt stress). (B) Word cloud showing enrichment of GO or KEGG terms for DEGs under salt stress. Red terms are associated with floe1-1 upregulated DEGs, black terms are associated with wildtype upregulated (or floe1-1 downregulated) DEGs. Font size is proportional to—log10(p-value). The only KEGG pathway enriched for the WT was “ribosome” (p-value=3.88E-17, not shown). (C) floe1-1 seeds show a decreased germination potential upon aging. Mean−+SEM. Four-parameter dose-response fit. Two-way ANOVA, ** p-value<0.01.

### DETAILED DESCRIPTION

“Modulating” seed germination as used herein refers to modulating the percentage of FLOE1-modified seeds that germinate in a given time frame compared to control wildtype seeds maintained under the same conditions, e.g., drought. Similarly, “modulating” seed viability (“viability” may also be referred to herein as “longevity”) refers to modulating the percentage of FLOE-1 modified seeds that are viable after a period of time, e.g., 1, 2, 3, 4, or 5, or more years, compared to control wildtype seeds maintained under the same conditions. Viability and germination can he assessed using routine methods. In some embodiments, germination and viability are assessed using methodology as shown in the examples.

Modifications to FLOE1 that influence germination rates include modulating the levels of expression of wildtype and mutant FLOE1. For example, decreasing the level of endogenous FLOE1 results in increases in germination rates under certain environmental conditions, such as drought, whereas increasing the level of expression of a wildtype FLOE1 decreases germination rate under certain environmental conditions, such as drought. In some embodiments, seeds having decreased endogenous FLOE1 expression will germinate faster, compared to control, under normal growth conditions. In some embodiments, seeds having increased levels of a wildtype FLOE1 remain viable longer compared to control, wildtype seeds.

An illustrative FLOE 1 sequence is provided below:

Arabidopsis thaliana FLOE1 (including the starting methionine):

MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQFMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGFRGDHVMAVIQRMEESGQPIDFNTL
LDRLSGQSSGGPPRGW

Domains include:

The DS-Rich Domain (DS Domain (Shown without the Start methionine)):

ASGSSGRVNSGSKGFDFGSDDILCSYDDYTNQDSSNGPHS
DPAIAASNSNKEFHKTRMARSSVFPTSSYSPPEDSLSQDI
TDTVERTMKMYA

### Nucleation Domain:

DNMMRFLEGLSSRLSQLELYCYNLDKTIGEMRSELTHAHE
DADVKLRSLDKHLQEVHRSVQ

### Coiled-Coil Domain:

ILRDKQELADTQKELAKLQLV

### QPS-Rich Domain (Short: QPS Domain):

QKESSSSSHSQHGEDRVATPVPEPKKSENTSDAHNQQLAL
ALPHQIAPQPQVQPQPQPQQHQYYMPPPPTQLQNTPAPVP
VSTPPSQLQAPPAQSQFMPPPPAPSHPSSAQTQSFPQYQQ
NWPPQPQARPQSSGGYPTYSPAPPGNQPPVESLPSSMQMQ
SPYSGPPQQSMQAYGYGAAPPPQAPPQQTKMSYSPQTGDG
YLPSGPPPPSGYANAMYEGGRMQYPPPQPQQQQQQAHYLQ
GPQGGGYSPQPHQAGGGNIGAP

### Domain of Unknown Function (DUF1421):

PVLRSKYGELIEKLVSMGFRGDHVMAVIQRMEESGQPIDF
NTLLDRLSGQSSGGPPRGW

Domains were defined based on their disorder scores or previous annotations. There are three structured regions: the nucleation domain, coiled-coil and DUF1421. The other two regions are highly disordered and were named based on their amino acid profiles: the DS-rich domain is enriched in D and S amino acids and the QPS-rich is rich in Q, P and S amino acids. Domains of a native FLOE1 polypeptide of a plant can be identified as described herein. Illustrative domain sequences of FLOE1 homologs are shown in FIG. 13. Homologs from across the plant kingdom show extensive sequence variation in both the DS and QPS disordered domains, but high conservation in the other domains. In some embodiments, a FLOE1 polypeptide has a nucleation domains, coiled-coil domain and DUf1421 domain, each domain having at least 70%, 75%, 80%, 85%, 90%. or 95% to the corresponding domain of an illustrative naturally occurring FLOE1 polypeptide sequence described herein. In some embodiments a mutated FLOE1, e.g., comprising mutations as described herein to modulate activity, has at least 60%, 65%, 70%, 75%, 80%, 85%, 90%, or 95% amino acid sequence identity to a naturally occurring FLOE1 polypeptide, e.g., any one of the FLOE1 polypeptide sequences as described herein. Percent identity can be determined by manual alignment, e.g., of short domains, or by using an algorithm, e.g., BLASTP.

In some embodiments, germination rates are modulated by mutating FLOE1, e.g., as described herein. In some embodiments, seeds are modified to remove all or a substantial portion of (e.g., removal of at least 60%, 70%, 80%, 90% or greater), of the QPS or DS domain, resulting in faster germination of seeds, e.g., under stress conditions such as drought.

In some embodiments, the levels of natural splice variants may be modified to modulate seed germination. For example, in some plants, a splice variant in which the DS domain is partly truncated can be up-regulated to enhance seed germination rates.

In some embodiments, seed gemination is modulated by introducing amino acid substitutions in FLOE1. For example, QPS has regularly spaced aromatic tyrosine residues along its sequence. In sonic embodiments, tyrosine residues in the QPS domain may be substituted with serine residues in multiple positions (see, e.g., FIG. 9). In sonic embodiments, tyrosine residues may be substituted with phenylalanine residues. In some embodiments, tyrosine residues may be substituted with tryptophan residues. in some embodiments, the DS domain may be mutated, e.g., to introduce substitutions, e.g., asparagines, at multiple aspartic acid positions.

Plants may be modified to introduce mutations and/or to increase or decrease FLOE1 expression using various techniques, including gene editing techniques. Exemplary genome editing proteins include targeted nucleases such as engineered zinc finger nucleases (ZFNs), transcription-activator like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 endonuclease to the target cleavage site. Alternatively, gene expression may be modified using interfering RNA, antisense or other methodology to reduce expression; or by overexpressing a gene to enhance expression.

Illustrative mutant FLOE1 sequences are provided below:

>FLOE1_ΔDS
MDNMMRFLEGLSSRLSQLELYCYNLDKTIGEMRSELTHAH
EDADVKLRSLDKHLQEVHRSVQILRDKQELADTQKELAKL
QLVQKESSSSSHSQHGEDRVATPVPEPKKSENTSDAHNQQ
LALALPHQIAPQPQVQPQPQPQQHQYYMPPPPTQLQNTPA
PVPVSTPPSQLQAPPAQSQEMPPPPAPSHPSSAQTQSFPQ
YQQNWPPQPQARPQSSGGYPTYSPAPPGNQPPVESLPSSM
QMQSPYSGPPQQSMQAYGYGAAPPPQAPPQQTKMSYSPQT
GDGYLPSGPPPPSGYANAMYEGGRMQYPPPQPQQQQQQAH
YLQGPQGGGYSPQPHQAGGGNIGAPPVLRSKYGELIEKLV
SMGERGDHVMAVIQRMEESGQPIDENTLLDRLSGQSSGGP
PRGW
>FLOE1_Δnucl
MASGSSGRVNSGSKGFDEGSDDILCSYDDYTNQDSSNGPH
SDPAIAASNSNKEFHKTRMARSSVFPTSSYSPPEDSLSQD
ITDTVERTMKMYAILRDKQELADTQKELAKLQLVQKESSS
SSHSQHGEDRVATPVPEPKKSENTSDAHNQQLALALPHQI
APQPQVQPQPQPQQHQYYMPPPPTQLQNTPAPVPVSTPPS
QLQAPPAQSQFMPPPPAPSHPSSAQTQSFPQYQQNWPPQP
QARPQSSGGYPTYSPAPPGNQPPVESLPSSMQMQSPYSGP
PQQSMQAYGYGAAPPPQAPPQQTKMSYSPQTGDGYLPSGP
PPPSGYANAMYEGGRMQYPPPQPQQQQQQAHYLQGPQGGG
YSPQPHQAGGGNIGAPPVLRSKYGELIEKLVSMGERGDHV
MAVIQRMEESGQPIDENTLLDRLSGQSSGGPPRGW
>FLOE1_ΔCC
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQQKESSS
SSHSQHGEDRVATPVPEPKKSENTSDAHNQQLALALPHQI
APQPQVQPQPQPQQHQYYMPPPPTQLQNTPAPVPVSTPPS
QLQAPPAQSQFMPPPPAPSHPSSAQTQSFPQYQQNWPPQP
QARPQSSGGYPTYSPAPPGNQPPVESLPSSMQMQSPYSGP
PQQSMQAYGYGAAPPPQAPPQQTKMSYSPQTGDGYLPSGP
PPPSGYANAMYEGGRMQYPPPQPQQQQQQAHYLQGPQGGG
YSPQPHQAGGGNIGAPPVLRSKYGELIEKLVSMGFRGDHV
MAVIQRMEESGQPIDENTLLDRLSGQSSGGPPRGW
>FLOE1_ΔQPS
MASGSSGRVNSGSKGEDEGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVPVERSKYGELIEKIVSMGFRGDHVM
AVIQRMEESGQPIDENTLLDRLSGQSSGGPPRGW
>FLOE1_ΔDUF
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQEMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAP
>FLOE1_8XY/F-S
MASGSSGRVNSGSKGSDSGSDDILCSSDDSTNQDSSNGPH
SDPAIAASNSNKESHKTRMARSSVSPTSSSSPPEDSLSQD
ITDTVERTMKMSADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQFMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGERGDHVMAVIQRMEESGQPIDENTL
LDRLSGQSSGGPPRGW
>FLOE1_8xY-S
MASGSSGRVNSGSKGEDEGSDDILCSYDDYTNQDSSNGPH
SDPAIAASNSNKEFHKTRMARSSVEPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQSYM
PPPPTQLQNTPAPVPVSTPPSQLQAPPAQSQFMPPPPAPS
HPSSAQTQSFPQSQQNWPPQPQARPQSSGGYPTSSPAPPG
NQPPVESLPSSMQMQSPYSGPPQQSMQASGYGAAPPPQAP
PQQTKMSSSPQTGDGYLPSGPPPPSGSANAMYEGGRMQSP
PPQPQQQQQQAHYLQGPQGGGSSPQPHQAGGGNIGAPPVL
RSKYGELIEKLVSMGERGDHVMAVIQRMEESGQPIDENTL
LDRLSGQSSGGPPRGW
>FLOE1_15xY-S
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQSSM
PPPPTQLQNTPAPVPVSTPPSQLQAPPAQSQEMPPPPAPS
HPSSAQTQSFPQSQQNWPPQPQARPQSSGGSPTSSPAPPG
NQPPVESLPSSMQMQSPSSGPPQQSMQASGSGAAPPPQAP
PQQTKMSSSPQTGDGSLPSGPPPPSGSANAMSEGGRMQSP
PPQPQQQQQQAHSLQGPQGGGSSPQPHQAGGGNIGAPPVL
RSKYGELIEKLVSMGFRGDHVMAVIQRMEESGQPIDENTL
LDRLSGQSSGGPPRGW
>FLOE1_5xS-Y
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESYSYSHSQHGEDRVATPVPEPK
KYENTSDAHNQQLALALPHQIAPQPQVQPQPQPQQHQYYM
PPPPTQLQNTPAPVPVYTPPSQLQAPPAQSQFMPPPPAPS
HPSSAQTQSFPQYQQNWPPQPQARPQYSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGERGDHVMAVIQRMEESGQPIDENTL
LDRLSGQSSGGPPRGW
>FLOE1_15xY-F
MASGSSGRVNSGSKGEDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQFFM
PPPPTQLQNTPAPVPVSTPPSQLQAPPAQSQFMPPPPAPS
HPSSAQTQSFPQFQQNWPPQPQARPQSSGGFPTFSPAPPG
NQPPVESLPSSMQMQSPFSGPPQQSMQAFGFGAAPPPQAP
PQQTKMSFSPQTGDGFLPSGPPPPSGFANAMFEGGRMQFP
PPQPQQQQQQAHFLQGPQGGGFSPQPHQAGGGNIGAPPVL
RSKYGELIEKLVSMGERGDHVMAVIQRMEESGQPIDENTL
LDRLSGQSSGGPPRGW
>FLOE1_4xY-W
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQWYM
PPPPTQLQNTPAPVPVSTPPSQLQAPPAQSQFMPPPPAPS
HPSSAQTQSFPQYQQNWPPQPQARPQSSGGYPTWSPAPPG
NQPPVESLPSSMQMQSPYSGPPQQSMQAYGYGAAPPPQAP
PQQTKMSWSPQTGDGYLPSGPPPPSGYANAMYEGGRMQWP
PPQPQQQQQQAHYLQGPQGGGYSPQPHQAGGGNIGAPPVE
RSKYGELIEKLVSMGFRGDHVMAVIQRMEESGQPIDENTL
LDRLSGQSSGGPPRGW
>10xD-N
MASGSSGRVNSGSKGENFGSNNILCSYNNYTNQNSSNGPH
SNPAIAASNSNKEFHKTRMARSSVFPTSSYSPPENSLSQN
ITNTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQEMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGFRGDHVMAVIQRMEESGQPIDENTL
LDRLSGQSSGGPPRGW
>FUS-DS
MASNDYTQQATQSYGAYPTQPGQGYSQQSSQPYGQQSYSG
YSQSTDTSGYGQSSYSSYGQSQNTGYGTQSTPQGYGSTGG
YGSSQSSQSSYGQDNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQEMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGFRGDHVMAVIQRMEESGQPIDENTL
LDRLSGQSSGGPPRGW
>QPS-DS
QKESSSSSHSQHGEDRVATPVPEPKKSENTSDAHNQQLAL
ALPHQIAPQPQVQPQPQPQQHQYYMPPPPTQLQNTPAPVP
VSTPPSQLQAPPAQSQFMPPPPAPSHPSSAQTQSFPQYQQ
NWPPQPQARPQSSGGYPTYSPAPPGNQPPVESLPSSMQMQ
SPYSGPPQQSMQAYGYGAAPPPQAPPQQTKMSYSPQTGDG
YLPSGPPPPSGYANAMYEGGRMQYPPPQPQQQQQQAHYLQ
GPQGGGYSPQPHQAGGGNIGAPDNMMRFLEGISSRLSQLE
LYCYNLDKTIGEMRSELTHAHEDADVKLRSLDKHLQEVHR
SVQILRDKQELADTQKELAKLQLVMASGSSGRVNSGSKGE
DEGSDDILCSYDDYTNQDSSNGPHSDPAIAASNSNKEFHK
TRMARSSVFPTSSYSPPEDSLSQDITDTVERTMKMYAPVL
RSKYGELIEKLVSMGERGDHVMAVIQRMEESGQPIDENTL
LDRLSGQSSGGPPRGW

### FLOE1 Homologs

In some embodiments, homologs are defined based on whether they contain an annotated DUF1421 domain. FLOE1 homologs can also exhibit conserved variation in their disordered domains. Illustrative homolog sequences are provided below:

Arabidopsis thaliana FLOE1 (FIG. 4D)
MASGSSGRVNSGSKGFDFGSDDILCSYDDYTNQDSSNGPH
SDPAIAASNSNKEFHKTRMARSSVFPTSSYSPPEDSLSQD
ITDTVERTMKMYADNMMRFLEGLSSRLSQLELYCYNLDKT
IGEMRSELTHAHEDADVKLRSLDKHLQEVHRSVQILRDKQ
ELADTQKELAKLQLVQKESSSSSHSQHGEDRVATPVPEPK
KSENTSDAHNQQLALALPHQIAPQPQVQPQPQPQQHQYYM
PPPPTQLQNTPAPVPVSTPPSQLQAPPAQSQFMPPPPAPS
HPSSAQTQSFPQYQQNWPPQPQARPQSSGGYPTYSPAPPG
NQPPVESLPSSMQMQSPYSGPPQQSMQAYGYGAAPPPQAP
PQQTKMSYSPQTGDGYLPSGPPPPSGYANAMYEGGRMQYP
PPQPQQQQQQAHYLQGPQGGGYSPQPHQAGGGNIGAPPVL
RSKYGELIEKLVSMGFRGDHVMAVIQRMEESGQPIDFNTL
LDRLSGQSSGGPPRGW
Dunaliella salina FLOE2L (FIG. 12B)
MDDMFEDLLAPPKKQPDPPPATTQQQQGTPEGGSSENGCV
KQQQKEGGDGKDAEQQPPAPGLVGVSKEELQSLVSVAVEG
AMDNLLGKFVKSLRLVLEDLGKRVDQQGTRLDSHSNEMKG
ALGEVLEQLESQAQNVHSRFTTVDMALKEVDRGVQALRDK
QELMEAQATLARFSHTDAAPQQQQQQQQQKPGAGAPPAVK
QEPAEPAPAAAAAPAAAPAPASSPSPAPAPAPTAAPASTP
AVPLPQPFPTQAGLPHQYAAPGAAPPHMPPYHQQAPSQAA
AALAPGAVPPHMLPPEPSAQYGGQPMQAYAGYNQPMPHAS
AVPPSSSPGPELAAAHSLPAYSQPMPAGYSQQPPTAPFPQ
PPQPMPMQPPQQFPPGAPYMPPTQPYGLHPSGSSGNLSMH
AGPAPSPILGPRYPAPLSYPAPPVAPAAYRPGGGSVSQGP
PSATRTSTRSVPVENIINDIAQMGFDRRQIMSVIADMQRE
GKAIDLNVVISRCLGS
Glycine max 2 (Gma-FLOE2L) (FIG. 4H)
MNTTPFMDKQIMDLTHGHGSSSSSTTQSQSKDFIDLMKEP
PQHHHHHHLEDEDNDEEEKARGNGISKDDIVPSYDFQPIR
PLAASNNFDSAAFSRPWNSDSNSNASPPVIKNYSSLDSME
PAKVIVEKDRSAFDATMLSEIDRTMKKHMENMLHVLEGVS
ARLTQLETRTHHLENSVDDLKVSVGNNHGSTDGKLRQLEN
ILREVQSGVQTIKDKQDIVQAQLQLAKLQVSKIDQQSEMQ
TSAITNPVQQAASAPVQSQPQLPTPANLPQSIPVVPPPNA
PPQPPPQQGLPPPVQLPNQFSQNQIPAAPQRDPYFPPPVQ
SQETPNQQYQMPLSQQPHAQPGAPPHQQYQQTPHPQYPQP
APHLPQQQPPSHPSMNPPQLQSSLGHHVEEPPYPPQNYPP
NVRQPPSPSPTGPPPPPQQFYGTPTHAYEPSSSRSGSGYS
SGYGTLSGPVEQYRYGPPQYAGTPALKPQQLPTASLAPSS
GSGYPQLPTARVLPQAIPTASAVSGGSGSTGTGGRVSVDD
VVDKVATMGFPRDHVRATVRKLTENGQSVDLNAVLDKLMN
DGEVQPPRGWFGR
Selaginella moellendorffii (Smo-FLOE2L)
(FIG. 4H)
MDNQGMGSHSEPFFDLLQPNTPSIAHASGSSSSNYVQNGP
RRMDSSPTYSFNNDDVLPSYDFQPLRSNGSGGGARIEEAG
GKFRQANPSFEQQVRDPPVTYEKYESTRSRHEFDKDAYDS
ATAAAVERTMKKYADNLLRVLEGMGGRLSQLEAATQRLEV
AFEKSKSANANNHGETDGRLRMLENMLREVQRGVQVVRDK
QEINEAQFQLKLQQDKTEAPTTKVEVQAPPVASSPQQPPP
MPQPPQALDSSVHQQQAPPPPPPLPVVHQPPPPTHIQQSP
HPPQHVPHAIQQQQQQPSYSYPPQNPAAPPPPPPPPMQQP
HPQPYPHQPEAPPYPPAPVPVSHPQGPPHHSQAPPVNYSL
DIPSYMPPPPPPQSYGAPPPPPPRQHQQQQQQQQHGPPPP
QMYDSLPGRTGSGPLALPPPPSAYQQQSYETSGYGGGGVN
YGRMHSGGGGGGGGGYPHLPTAQPIQQSLPSARPASRSGV
DDVIDKVAAMGFPRDQVRATVQRLTENGQAVDMNVVLDKL
MNGGGSDAGPPKAGWFGR
Wollemia nobilis Wno-FLOE1L (FIG. 4H)
MEHQELGEGKENFLGFAPSGSSNPPSVNGNPSISRSGYKV
TEGSAPGFDFSSEDILSSYEYNKKQNFSDGHYVAPSRLSN
FPSDSYLNSSRSDRFRESRTAKPYANEQSQEDDNRYNEIV
GTVERTMKKYADNLLKVLDGMSNRLMQLELVNERLERSVG
EMRADMAEDHKENGERFRMLEDHVHEVHRTIQILRDKQEI
AEAQTELAKLQLARKESSSNFQSPEDKTLTSSTLSEVKKE
HAFQPQNVQAQLRSSNPAFPALPALPAPPQSSPSPSLPMP
AREQCQSLLPQQQQPAQVSMVQQSPVTSFPLQQVAQLPQQ
PNVMLMQPYYPQQQGQIQPVPQAPQAGQVPHIQQQPPQPA
VAAPPQVQNLPYGCQPQHIQNIPNQSSQHVQRPQIQQMPR
LQSQPPPQTQMQPQPLSQQPHLPQQAQMRPNIYSGQTHGV
PPEAFAYAPETGQHQTQAPYQGGPSSIPSEASMYNYGGPP
QIIQPSSQGQVSIQSHRPQYPPSDSSNASSALVPPPVGHP
MHGYSAYNSPPRPAPSPYGVPFSGAPQTTPFPGAYMRFPS
AQQQYAHPSGNAVPNTSGGHLPSSHAFDDLVEQVATMGFS
RDQVRVTIQQLTESGQPVDMNSVLDRLNNSPGPSQRGWYN
Theobroma cacao Tca-FLOE1L (FIG. 4H)
MASGSSGRGNSGGSKGFDFGSDDILCSYEDYGNQESSNGS
HAEPVVGTNSSAKDFHKGRAARSIFPPNAYSQPEDSFSTD
VTATVEKTMKKYADNLMRFLEGISSRLSQLELYCYNLDKT
IGEMRSDLVRDHVDADLKLKSIEKHLQEVHRSVQILRDKQ
ELAETQKELAKLQLVQKESSSSSHSQSTEERASPPASDSK
KTDHTSDMQSQQLALALPHQVAPPQQPVVPHSQASPQNLT
QQSYYIPPNQLSNSQAQVQAPAPAPVPTPAPAPAPAPIQH
PQSQYLPSDSQYRTPQIPDISRMPPQPTQSQVNQVPPVQS
FPQYQQQWPQQLPQQVPQQQSSMQPQMRAPSTPAYPPYPP
TQSTNPSLPEALPNSLPMQVPYSGVPQPVSSRADTIPYGY
GLPGRTAPQQPQQIKGTFGAPPAEGYTAPGPHPPLPPGSA
YMMYDSEGGRPLHPPQQPHFSQGGYSPANVSLQTPQTGTG
PNVMIRNTSHSQFIRSHPYSDLIEKLVSMGFRVDHVASVI
QRMEESGQPVDFNAVLDRLNVHSSGGSQRGGW
Marchantia polymorpha (Mpo-FLOE2L)
(FIG. 4H)
MDSSLGIGTNHQPGAQNEPFFDLLQPAVTSSSSLGQNPPQ
NSSKMENSGEFNFSDDVLPSFDFQPIRTSGAPPLKTSNSG
AGRMEESRSRQASPPPSYSSYEPMVRRSREPPPTYEAPLP
RSQEHEKESFETATVAAVERTMKKYADNLLRVLEGMSGRL
SQLESSTQRLEELYGEIRNDVVNNHGEVDGKLRSLENHIL
EVQRGVQLLRDRQELAEAQSQLAKLQAVTKSDVAPHNSAP
SAPPPVIEQLPELSRASSGKALLEDSQQQMSNVASSHYQQ
PQPQHLQQLQLQLPSVPSHSLPQPLPQQQQQPQPQAQQHQ
PQQQQRNPSKKKGKGGVHQGPQMQQQSEVSHQILQQQQQQ
QQPPPPPPPPQQMSHSQHSPPPPPPPPPSQMTMPFYSQQQ
QPLPQAPPPMPTYGHQPEAPAYNQHPQGPHHVPPTPQSYP
SDLPSYHPSNYGPPGSGLAQPPRQSSQIPPSSHIQQHHNV
PMYDPSLARNGSGQLALPPPYLPQAQQVSNSPIYEPQSPG
SGYPSSSYRVAQPVPSAPSGGGYPRLPVAQPLPHAMPAGG
SGGGPPGTPPLSTNRVPIDEVIDKVTAMGFSKDQVRAVVR
RLTENGQSVDLNIVLDKLMNGGDAQPPPKGWFGR
Chlamydomonas reinhardtii (Cre-FLOE2L)
(FIG. 4H)
MEDDLFGDLLGGPKPKPSNLTSPTGTASKDGHAGKAKTSA
ASANGADEEASGSGAATRSENAEKVTLSADDLAALVDKGV
HAAMEATFSKFVRSLRTVLEDMTRRVSAQDVTLAELRHSV
DELRDTVAAQPADLHIRFSNLDTAFKEVERNVQGIRDKLE
LQEAQALLAQMSSDVRAKGSSTSSAGAAPAAAAAPEAAAA
PAAASAPAPAPAAAAPAAAPVAPAPAAAPAPAPVAQQAPV
APQAPMPAPVTQQAPAVGAPMPGMQYGAPQQQQAPQLQQQ
QQQQQQPQQQQQQLPPHMQPYGAPAPAPGMPGAPPLPMQP
QQLQLQQQPSMEAKPVMQQPQQQQQQPQQQPYGAPGYPQY
QQQPQQMPPPGVPDQGHYGAPAALPGPAPGGYPAGPYGGM
PPQEAPRAPVMPQQHMAPPHMGVPPPAAAPRMDHPPPGAP
PPPGMAYPAPPAMHAYPPPPAVPSYGRPQAAPPPTYRSPM
PGPGPVSAPPGPPGGAPGGPPGTASRTVPLDQIIADIAQM
GFSRGDVLNAVNNLQMSGKALDLNTIIDKLTRG
Klebsormidium nitens (Kni-FLOE2L) (FIG. 4H)
METNKGGKYPAPSFSTENEPFYDLLKTGNNANQQSSLSGV
ATNPVDFGENILPSYDFHPTRPAPSLNNGNKMMSPTLSEQ
SLDGKSSTSEPLHGKQERSVADVDDSKDAVAAVERTMKKY
ADNLLRVLEDMRGKLTQLERTTDRLESTVAELQNRSADQH
GELDGRVRGLEHVLREVQRGVQLLRDKSELQEAQAELAKM
QMTTTAAKPPLPAQAPPALTAPPQTFPALTAPPLVPEEPA
KPAAPMQMQPQPQVEQQPAPAPVPLPSAPSAPPQQLSVPV
PQYQAPPKPPASPHPRHPPQPQQPQGPSGPAPRPRQYGPQ
APPYMQRPPPQQQQEAPAYLPQGYGQQAGAPPHQMPPPPP
QPQQGPPRQGYEGAPPQGAPHPGGRLALPPPPGSYGPPPP
QGYSERPGSTGGYDRPPSASYDRPPTSGYERQAPPPFERP
PPPNYDRQSGYEPRVPASPYGPPPQYGAGGPPPAPGTYPR
LQMAQPVQSSEPPRTAGSGGPAQLSTSKMPIEQVIDDVAA
MGFHKDEVRSIVRQLTETGKSVDLNIVLDTLMTRSGGAAP
TGRSW
Bathycoccus prasinos (Bpr-FLOE2L) (FIG. 4H)
MEDDDPFDFKIGVEKNALNSGKKTTTEAMMKSMMMKPSST
TLESSSFTSFGEEEKKTMTMNDGVKGIPESKAPSSTKTDE
DQKKKKNDDDDDAKVNATIESFSTETKVILTTLGKILERL
EALELVALRNAKEVARVENALHGFIVGQARKENGKEPITS
ANLFAVVDSSEEEEEEEEEEEKIEEEIKENIVLRAGSGRS
RRPPTPEGAHHPPHYPPHNPPPHHPPPPHAHHQHHQDPYG
PPSFARGGRGGPPHPHPPPPPHERSGSPSGESAAHYPGID
HHLHPHPHRSPPPPHHGGPSSPPPHHGHPPPPSHVQHDPY
GTYHPSPPPPPQVLASSYPSPPPPSPPQVQNEDIPLDVIV
GEFASMGFTRDEVMTVLGKMEARNEQKEMNSILDKLMAGE
GKL
Solanum lycopersicum 2 Sly-FLOE2L (FIG. 4H)
MDLSTNNDFINLHDDQHHITAGVNHPVRPIESFPNCSIHW
APDTKTNTNYSSPDSIEPAKLIVEKDLSTIDASLLSEIDH
TVKKYADNLLHAIESVSARLSQLETRSRQIEDFVVKLKLS
VDNNHGNTDGKLRLVENILREVQDGVQVIKNKQDIMETQL
QLGKLQVPKEIDSSIVDSAHHRASAPLQSHQQFPPVVLAQ
PPSPLPPPNAPPPPLQQKIPSQVELQDQFPQNLIPSGTQR
ETYFPLTGQAPENSSQQNQQSAPHQRLQTSIPPPPHQQYL
PFPSSLYTQPPVPSQAHSPLPSVNPSQSQPPLIHHPEERH
FIASQTYPQANTSQFPSHPSSGAPVSHHFYAAPANLFEPP
SSRQGSGFSSAYGPSTGPGESYPYSGSTVQYGSGSPFKSQ
QLASPLMGQSGGNGYPQLPTTRILPQALPTAFAVSSGSSS
PRTGNRVPIDDVVDKVINMGFPRDQVRATVQRLTENGQSV
DLNVVLDKLMNGG
Coffea canephora FLOE1L (FIG. 12B)
MASGSAGRPSNSGSKPFNFVSDDILCGPYEDYGNQDGSNG
TSHSDPAIGATSAKEFHKNRMARSSVFPAASYSPPEESSF
NQDVIATVERTMKKYADNLMRFLEGISSRLSQLELYCYNL
DKSIAEMRSELGGDHTEAETKLKSLEKHLQEVHRSVQILR
DKQELAEAQKELAKLHLAQKESSSASNLPQKEERVSAPAS
DAKKSENSSDSHGQQLALALPHQVPQPQQQQPPSVAPPPP
MPSQSVPQAQAYYLPPHQLPNVPAAASQPSQGQYLPPDSH
YRAPQLQDVSRVAPQPAQSQVNQAPQVQTIPSYQPQWPQQ
LPQQVQPLPQQSVQPQIRPSSPPVYSSYLPNQANPPPPEA
LPNSMPMQVPFSGISQPGPVRAETVPYGYGGAARPVQPQP
QPQHLKATYASPADGYAASGPHPTLSPGNTYVMYDEAGRP
HHPAQQPHFPQSPYPPTTMPPQNLQPNTGSNLVVRPPQFV
RNHPYGDLIEKVVSMGYRGDHVVSAIQRLEESGQPVDFNA
VLDRLNGHSAGGPQRGWSG
Arabidopsis thaliana FLOE2 (FIG. 4D)
MQSFDLIKSALFSDKQIMDLMNDNSNNSQDGDHQNYRVGD
NGLESKKEAIFPSYDFQPMRPNASAGLSHHALDLAGSVNS
TAARVWDASDPKPVSASSARSYGSMDSLEPSKLFAEKDRN
SPESAIISAIDRTMKAHADKLLHVMEGVSARLTQLETRTR
DLENLVDDVKVSVGNSHGKTDGKLRQLENIMLEVQNGVQL
LKDKQEIVEAQLQLSKLQLSKVNQQPETHSTHVEPTAQPP
ASLPQPPASAAAPPSLTQQGLPPQQFIQPPASQHGLSPPS
LQLPQLPNQFSPQQEPYFPPSGQSQPPPTIQPPYQPPPPT
QSLHQPPYQPPPQQPQYPQQPPPQLQHPSGYNPEEPPYPQ
QSYPPNPPRQPPSHPPPGSAPSQQYYNAPPTPPSMYDGPG
GRSNSGFPSGYSPESYPYTGPPSQYGNTPSVKPTHQSGSG
SGAYPQLPMARPLPQGLPMASAISSGGSGGGSDSPRSGNR
APVDDVIDKVVSMGFPRDQVRGTVRTLTENGQAVDLNVVL
DKLMNGDRGAMMQQQQQQPPRGWFGGR
Arabidopsis thaliana FLOE3 (FIG. 4D)
MNTCQFMDKQIMDLSSSSSLPSTDFIDLMNNHDGDDHQKK
QVIGDNGLDSKKEVIVPSYDFHPIRPTTAARLSHSALDLA
GSTTRVNWSASDYKPVSTTSPNTNFGSLDSIEPSKLVPDK
GQNVFNTTIMSEIIDRTMKKHTDTLLHVMEGVSARLSQLE
TRTHNLENLVDDLKVSVDNSHGSTDGKMRQLKNILVEVQS
GVQLLKDKQEILEAQLSKHQVSNQHAKTHSLHVDPTAQSP
APVPMQQFPLTSFPQPPSSTAAPSQPPSSQLPPQLPTQFS
SQQEPYCPPPSHPQPPPSNPPPYQAPQTQTPHQPSYQSPP
QQPQYPQQPPPSSGYNPEEQPPYQMQSYPPNPPRQQPPAG
STPSQQFYNPPQPQPSMYDGAGGRSNSGFPSGYLSEPYTY
SGSPMSSAKPPHISSNGTGYPQLSNSRPLPHALPMVSAVS
SGGGSSSPRSESRAPIDDVIDRVTTMGFPRDQVRATVRKL
TENGQAVDLNVVLDKLMNEGGAPPGGFFGGR
Physcomitrella patens (Ppa-FLOE2L) (FIG. 4H)
MLVDQMEYQGQQGSGGPQDDAFYELLSSTALANAKKQQQQ
QHQFEQQNHQQQQQQQFDSRSEEGLPNYDFQSTSSSYGGV
VANGEDMRKAPSVMPVVESSHPPHFPTYPPGSSYSNARQH
LPVPSFVESSPPRQEKGNAEAATVAAVEQTMKKYADDLMR
MMESMAGRIGQLESSTRRLEQIMTDFKGGSEKSQGVSGGK
LLLIETMLSEVQRGVQELRNKQEVMDAQSTIGKLQLGDEG
VSSSVHSQTSLEPPPAQSPRAPQMPETPPYPMGPLPHAPH
HPPGHLPPYMVPPQLVGLAPPPPPPPAPEPHYQPSQQGPP
PPPPPPPQQSYHSQQLQQQSTPPSAHPHGPFPQPPELPPY
GATPQGPYKGQSGSFGQDAPPPSYGGRPHHMPQTGLGGSQ
MYDQSGGIPPYQSQGRPAAPAYDQPIGLPPPGYFNPGYRS
GQQTPSAPSSGAGGYPRLPTAQPVQHAMPTAREREGAQPS
SGATPLSTNRLSIDEVIDKVAVMGFSKDQVRAVVRRLTEN
GQSVDLNVVLDKLMNGDGGAQPPKGWFQRG
Solanum tuberosum (Stu-FLOE1L) (FIG. 4H)
MASGSSGRPSNSSGSKGFDFGSDDILCSYEDYPHQDASNG
THSDPAIATNSAKEFHKNRMTRSSMFPTSTYSPPEESSFN
QDMICTVEKTMKKYTDNLMRFLEGISSRLSQLELYCYNLD
KSIGEMRSDLVRDHGEADLKLKALEKHVQEVHRSVQILRD
KQELAETQKELAKLQFAQKEPASANNSQQNEDRNAQPVSD
SNKGDNSTDVNGQELALALPHQVAPRAPLTNQPVEQPQQA
PPQPIPSQSMTQSQGYYLPPVQMSNPPAPTHLSQGQYLSS
DPQYRTSQMQDLSRLPPQPAAPPGNQTPQIQSMPQYQQQQ
WTQQVPQQIQASQQVQQHQLPTVQQQGRPSSPAVYPSYPP
NQPNPSPEPVPNSMPMQMSYSAIPQSVACRPEAIPYGYDR
SGRPLQSQPPTQHLKPSFGAPGDGYATSGPHPSLSAGNAY
LMYDGEGPRGHPSQPPNFPQSGYPPSSFPPQNAQSSPSPN
HMVRPPQLMRTHPYNELIEKLASMGYRGDHVVNVIQRLEE
SGQTVDFNTVLDRLNGHSSGGPQRGWSG
Solanum lycopersicum (Sly-FLOE1L) (FIG. 4H)
MASGSSGRSNNAGSKGFDFASDDILCSYEDYANQDPSNGT
HSDSVIAANSAKEFHKSRMTRSSMFPAPAYSPPEESSFNQ
DMICTIEKTMKKYTDNLMRFLEGISSRLSQLELYCYNLDK
SIGEMRSDLVRDHGEADSKLKALEKHVQEVHRSVQILRDK
QELAETQKELAKLQLAQKGSTSSSNSQQNEERSAQHLSDD
KKSDDAPEVHGQQLALALPHQVAPQMANQQAPTQLSQGQF
LSSDPQYRNPQMQVTPQRAAPQVNQTQQLQSMPQYQQQWA
QQVPQQVQQSQIPNMQQQARPASPAVYPSYLHSQPNPTPE
TMPNSMPMQVPFSGVSQPVASRPESMPYGYDRSGRPLQQQ
PATPHLKPSFGAPGDGYAASGAHPTLSPGNAYVMYDGEGT
RAHPPPQPNFQQSGYPPSSFPPQNQQPAPSPNLMVRPPQQ
VRNHPYNELIEKLVSMGYRGDHVVNVIQRLEESGQPVDFN
AILDRMNGHSSGGPQRGW
Glycine max (Gma-FLOE1L) (FIG. 4H)
MASGSSGRGNSASKGFDFASDDILCSYDDYANRDSTSNGN
HTDPDFHKSRMARTSMFPTTAYNPPEDSLSQDVIATVEKS
MKKYADNLMRFLEGISSRLSQLELYCYNLDKSIGEMKSDI
NRDHVEQDSRLKSLEKHVQEVHRSVQILRDKQELAETQKE
LAKLQLAQKESSSSSHSQSNEERSSPTTDPKKTDNASDAN
NQQLYLPSDQQYRTPQLVAPQPTPSQVTPSPPVQQFSHYQ
QPQQQQQPPQQQQQQWSQQVQPSQPPPMQSQVRPSSPNVY
PPYQPNQATNPSPAETLPNSMAMQMPYSGVPPQGSNRADA
IPYGYGGAGRTVPQQPPPQQMKSSFPAPPGEMYGPTGSLP
ALPPPSSAYMMYDGEGGRSHHPPQPPHFAQPGYPPTSASL
QNPPQGHNLMVRNPNQSQFVRNHPYNELIEKLVSMGFRGD
HVASVIQRMEESGQAVDFNSVLDRLSSVGPQRGGWSG
Sphagnum fallax FLOE2L (FIG. 12B)
MDAFGGASSGMGSVQTGSQNDVFYDLLSNSTSALNGGGQQ
KKRDLVETRVSSPVVDFGNEEVQPPRYDVQPSYDFQPSAS
ALGNSKITAFSSGNLSSSLRPPLTSEPTVHYEKEVIENAT
LVAVERTMKKYADNLLHVLEGISGRLTHLESTTQRLEHMV
TEFKGGADENSSATDGKLRALGNMLSEVQRSVQVLRDRQE
LAEAHSQLAKLQLSVREGAPSAPVATQAPEPRPQSPPPPR
HSDALPQQQGQSTSRHNPQLPTPPPHMLPQQPSPPLLPQQ
LQLQAPPAVQPEPQYQQQSPQPPPPHSMSFYSQPPPPPPP
PPPPQQQQGPPPSLQQQYSHPPEAPPYGTHPQGPHQGPPP
PSANYADLPPQFMPFGNRPFPQQQPPPMQTLQPQAGSGGP
PMYDTQAGGSSSSSMGLPPPYHSQGRPAVPNYDQQQMNAP
AGYGSPAYHRMPQPAVPSAPSSGNGGYPRLPTAQPVQHAL
PTATATGPGPSGPAPLSTNRVPIDEIIEKVSSMGFSKDQV
RAVVRRLTENGQSVDLNIVLDKLMNGGADVQPQKGWFGRG
Theobroma cacao 2 (Tca-FLOE2L) (FIG. 4H)
MNTSQFMDKQIMDLTSSSSSPPHNTNKDFIDLMNNPQNED
NHNQGSGISNKEGIFPSYDFQPIRPVSTSLDAAAVNNNPR
SWSSGDSKTKNYGSLDSVEPAKVILEKDRNAFDTSIVAEI
DRTMKKHTDNLIHMLEVVSARLTQLESRTRNLENSVDDLK
VSVGNNHGSTEGKMRQLENILNEVQTGVHVLKEKQEIMEA
QLHLAKLQVTKGDHPSETQNTVHVDTVQQAASAPFQSHQQ
LPPAASFPQSLPSVPPPPTVPPLVLPQQNLPPPVQHPNQF
PQSQVPSVPQRDAYYPPPGHTQEAPGQQFPVPPTQQPQLP
PAAPPHQPYQPVPPPQYSQPPQPVQLQPSLGHHPEEAPYV
PSQNYPPNLRQPPSQPPSGPPSSQQYYGAPPQMHEPPSSR
PGSGFSAGYIPQSGQSEPYAYGGSPSQYGSGSPMKMQQLP
SSPMGQSGGSGYPQLPTARILPHALPTASGVGGGSGPSGP
GNRVPVDDVIDKVTSMGFPRDHVRATVRKLTENGQSVDLN
VVLDKLMNDSEVQPPRGWFGR
Ostreococcus tauri (Ota-FLOE2L) (FIG. 4H)
MPSAREDIDPFDLLSPIASDARRRARAVTDEKTTATTTTG
TMTNESRSIRHADADADAVRDEAMEKLISRVEALERVSRD
GFARVGEVLERLTGRVETLSARVAAMRRDEEYDDEDSSDS
SGDEAEEASEDVREEDGYADVPRRRGSPPRRRRRSPPRHH
RGPPPPRRRGSPPPRHHRGSPPHHQHGPPPDHGGPPPHHH
HGPPPLDHRGPPPHHHGPPPPHHHGPPPHQHGPPPPPSYE
QMVPPTAYPSSPYPMYAPPPEPPRAPPPESPRSMAPPPVT
SGAVPLEQMIGDFANMGFTRQQVMNAVSEMASSGQKIEVN
SVLDRLMRAHA
Wollemia nobilis 2 (Wno-FLOE2L) (FIG. 4H)
MQQGPPNAMQISAYSQNPQPQQPSGQSVSIPFSQPEPTPS
LAQHMPHSQMPTPALPGNYGPEPPYMPSNYGGSSSHQPPR
SMPPPQLPASQRFSGSQQGYEPTFGRTSSGPLPFPPTYGP
GLSGPPPYGDSQTYSGPSFRLPQKDSNPSGGGSSAGHPRL
PTAKPLQHSLPVASSVNSSPSGSTSSSNRVPVDDVVDKVS
SMGFPRDQVKMVVQKLTENGQSVDLNVVLDKLMNGGGGEI
QPQKGWFGR

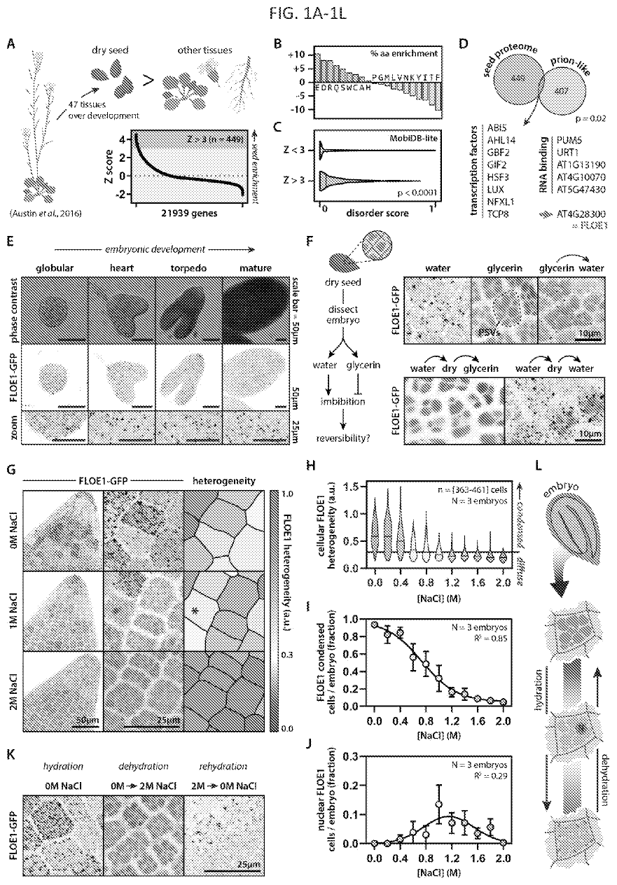

Because limited water availability dramatically alters protein solubility and plant seeds are known to undergo a cytoplasmic liquid-to-glass transition during maturation (3, 4), we investigated how plant seed proteins might have adapted to these extreme conditions (FIG. 1A). We re-analyzed existing Arabidopsis thaliana transcriptoinics data and found 449 protein-coding genes that are relatively more expressed in dry seeds compared to other tissues (FIG. 1A) (8, 9). Compared to the rest of the proteome, these seed proteins had a different amino acid profile (FIG. 1B, FIG. 5) and were enriched for regions of structural disorder (FIG. 1C). Intrinsically disordered proteins (IDPs) have emerged as key players orchestrating how cells organize themselves and their contingent biochemical reactions into discrete membraneless compartments by a process called liquid-liquid phase separation (LLPS) (10, 11). A subset of IDPs are proteins that harbor a prion-like domain (PrLD) and we identified 14 proteins with PrLDs enriched in the seed proteome (FIG. 1D). PrLDs share similarities to domains from fungal prions and can drive reversible protein phase separation in diverse eukaryotic species (12). In yeast, deploying these PrLDs is a powerful tool for generating phenotypic diversity to help cope with and survive in a fluctuating environment (13). All but one of these plant PrLD-containing seed-enriched proteins had annotated functions or domains related to nucleic acid metabolism. The one that did not, AT4G28300, was an uncharacterized plant-specific protein, which we named FLOE1.

FLOE1 accumulates during embryo development and its expression peaks in the mature desiccated state (FIG. S2). We generated transgenic Arabidopsis lines expressing FLOE1-GFP under control of its endogenous promoter and with its non-coding sequences intact, FLOE1 formed cytoplasmic condensates during embryonic development (FIG. 1E, FIG. 7A) and in embryos dissected from dry seeds (FIG. 1F, FIG. 7B). However, when we dissected dry seeds in glycerin instead of water (to mimic the desiccated envirommat) FLOE1 did not form condensates and was localized diffusely (FIG. 1F, FIG. 7C-D). When we transferred these embryos from glycerin to water, FLOE1 condensates spontaneously appeared (FIG. 1F) and were fully reversible with repeated hydration-dehydration cycles (FIG. 1F). We pre-treated seeds with the translation inhibitor cycloheximide and this did not affect the formation of FLOE1 condensates, indicating that they are distinct from stress granules and processing bodies (14), and that their emergence was not due to FLOE1 translation upon imbibition (FIG. 7E). To directly test whether FLOE1 forms condensates in response to changes in water potential, we incubated dissected embryos in solutions of varying concentrations of salt, mannitol, or sorbitol (FIG. 1G-K; FIG. 7F-G). High concentrations of salt resembled dry conditions and embryos lacked visible FLOE1 condensates (FIG. 1G-J). Lowering the salt concentration resulted in a gradual emergence of condensates, which was highly variable at the cell-to-cell (FIG. 1G-H) and tissue levels (FIG. 1I), following a switch-like behavior. Notably, in intermediate salt concentrations, we observed a small number of cells with apparent nuclear localization of FLOE1 (FIG. 1J), suggesting this could be a behavior associated with early steps of imbibition, before the majority of the protein condenses in the cytoplasm. Similar to our observations with repeated hydration-dehydration cycles, FLOE1 condensation was also reversible by moving the embryos back and forth between solutions of high or no salt (FIG. 1K). Thus, FLOE1 forms cytoplasmic condensates in response to changes in water potential (FIG. 1L).

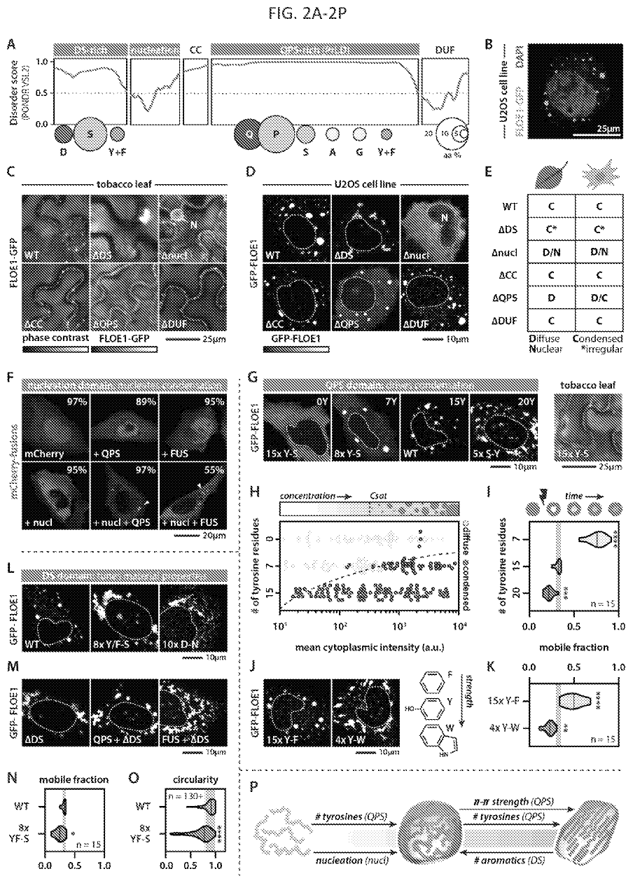

Numerous yeast proteins undergo oligomerization or phase separation upon stress-induced quiescence (15) but to our knowledge FLOE1 is the first example of a protein undergoing biotnolecular condensation upon release from the quiescent state. To define the mechanism by which FLOE1 undergoes this switch, we dissected the molecular grammar underlying this behavior. FLOE1 harbors a predicted short coiled-coil domain and a conserved plant-specific domain of unknown function (DUF1421) (FIG. 2A). Disorder prediction algorithms identified another predicted folded region and two different disordered regions, one enriched for amino acids aspartic acid and scrim (DS-rich) and the other enriched for glutamine, proline, and serine (QPS-rich). We heterologously expressed FLOE1 in two orthogonal systems, tobacco leaf (FIG. 2B-C, FIG. 8) and the human osteosarcoma cell line U2OS (FIG. 2D). In these two systems, as well as in Arabidopsis, FLOE1 formed spherical condensates, providing independent platforms for interrogating the molecular drivers of condensation. We systematically deleted each domain of FLOE1 and assayed the impact on cytoplasmic condensation (FIG. 2C-E). In both tobacco and human cells, mutants lacking; either the short coiled-coil domain or DUF1421 behaved identically to the wildtype protein (FIG. 2C-E). Deletion of the other domains altered FLOE1 condensation (FIG. 2C-E). Deletion of the predicted folded domain, which we refer to as the nucleation domain, abolished cytoplasmic condensation, resulting in a fraction of the protein redistributing to the nucleus. Folded oligomerization domains play important roles in nucleating phase separation of several IDPs (11). Indeed, expression of chimeric fusion proteins revealed that this domain is sufficient to nucleate phase separation of different PrLDs (FIG. 3F).

In line with their role in driving phase separation of other prion-like proteins, deletion of the QPS PrLD reduced condensate formation (FIG. 2C-E). Consistent with the emerging sticker-spacer framework for PrLDs (17, 18), the QPS PrLD has regularly spaced aromatic tyrosine residues along its sequence that may act as attractive stickers (FIG. 9). Substituting tyrosine residues for serines (Y-S) decreased condensate formation in both human and tobacco cells in a dose-dependent manner (FIG. 2G, FIG. 9). By mapping out a phase diagram (FIG. 2H) and probing the molecular dynamics using fluorescence recovery after photobleaching (FIG. 2I) of Y-S and S-Y mutants, we confirmed that the number of tyrosines determines both the saturation and gelation concentration of FLOE1 condensates, consistent with what has been shown for other PrLDs (18). These findings provide evidence that FLOE1 condensates form via LLPS, and increasing its multivalency drives gelation into more solid-like irregular assemblies. While changing the number of stickers can drive a liquid-to-gel transition, altering sticker strength may also alter the gelation concentration. Substituting tyrosines for weaker (phenylalanine) or stronger (tryptophan) aromatic residues affected both condensate morphology and intracondensate FLOE1 dynamics in a predictable manner (FIG. 2J-K, FIG. 9). While increasing the stickiness of the QPS PrLD induced gelation of FLOE1, this was also the case for deletion of the N-terminal DS domain (FIG. 2C-E, L). Surprisingly, serine substitution of aromatic residues in this domain had a similar effect as deleting the domain (FIG. 2L) and the mutated FLOE1 exhibited a mode solid-like behavior (FIG. 2N-O), which suggests that the aromatic residues in each disordered domain have opposing functions. Similarly to the 8×Y/F-S substitution, the 10×D-N mutant results in the formation of solid-like irregular assemblies, with the latter presenting with a more filamentous morphology (FIG. 2L). To test whether the presence of a PrLD would rescue the liquid-to-gel transition of the ΔDS mutant, we replaced the DS domain with sequences of the same length derived from the QPS PrLD and the FUS PrLD. Even though these domains have regularly spaced tyrosine groups, they still formed gel-like assemblies (FIG. 2M). This suggests that other amino acid residues in the DS domain contribute to its function, which is in line with our findings for the 10×D-N mutant. Thus, synergistic and opposing molecular forces tightly regulate FLOE1's biophysical phase behavior, and changing this balance allows us to toggle its properties between dilute, liquid droplet and solid gel states (FIG. 2P).

We next asked whether these various physical states of FLOE1 have a role in germination. Lines carrying the knockout allele floe1-1 did not show any obvious developmental defects, and floe1-1 seeds had the same size and weight as the wildtype (FIG. 10A). floe1-1 seeds germinated indistinguishably to the wildtype under standard conditions (FIG. 10B), but actually had higher germination rates under conditions of water deprivation induced by salt (FIG. 3A, FIG. 10C) or mannitol (FIG. 10C). We confirmed that these phenotypes were caused by mutations in FLOE1 using independent lines carrying CRISPR-Cas9 FLOE1 deletion alleles and floe1-1 lines complemented with the wildtype allele (FIG. 10C-F). Thus, FLOE1 is a dosage-dependent negative regulator of germination under water limitation. Germination during stressful environmental conditions is risky for a plant and can reduce fitness. Indeed seedlings displayed developmental defects or eventually died under these conditions (FIG. 3B, FIG. 10G), whereas ungerminated seeds retained full germination potential upon stress alleviation (FIG. 3C), in line with bet-hedging strategies in stressed seeds (19-21). Importantly, whereas ungerminated salt-stressed seeds were largely devoid of FLOE1 condensates, even after 15 days of incubation, alleviating salt stress induced their robust appearance (FIG. 3D, FIG. 10H). This shows that FLOE1 phase separates during physiologically relevant conditions in vivo. To directly test if FLOE1's function depends on its ability to undergo phase separation we generated complemented Arabidopsis lines carrying wildtype or different FLOE1 domain deletion mutants (FIG. 3E-F). These mutants behaved the same way in Arabidopsis embryos as they did in human and tobacco cells (FIG. 2C-E). The ΔQPS mutant was unable to phase separate upon imbibition (FIG. 3F), whereas the ΔDUF mutant formed condensates similar to wildtype (FIG. 3F-H). In contrast, the ΔDS mutant formed condensates that were much larger than those formed by wildtype (FIG. 3G-H), and also seemed to have lost some of their hydration-dependency (FIG. 3I), consistent with their solid-like biophysical properties. We assayed germination rates under salt stress and found that, whereas the DUF domain was dispensable for function, removing the QPS domain resulted in FLOE1 loss of function (FIG. 3J, FIG. 11A-D). In contrast, ΔDS complemented lines exhibited a greatly exacerbated germination rate under stress, surpassing even that of the floe1-1 null mutant, indicating that ΔDS likely functions as a gain-of-function mutation (FIG. 3J, FIG. 11A-D). Interestingly, even under standard conditions the ΔDS mutant displayed faster germination rates (FIG. 11C). In the evolutionary game theory framework, this ΔDS mutant behaves like a “high-stakes gambler” that perceives the risk of germination under stress (e.g., seedling dying) to be lower than the chance of a change in environment (e.g., increased rainfall). Thus, FLOE1 seems to function as a water stress-dependent “resistor” in the signaling cascade that triggers the initiation of germination upon imbibition, tuning bet-hedging strategies at this crucial step of a seed's life.

If FLOE1 acts as a molecular tuning knob, we predict there should be natural variation in its phase separation behavior. FLOE1 has an annotated shorter splice isoform that lacks the majority of the DS domain (FIG. 4A), which forms larger ΔDS-like condensates (FIG. 4B) that are able to recruit the longer isoform (FIG. 4C). Searching the Arabidopsis genome, we found two FLOE1 paralogy, FLOE2 (AT5G14540) and FLOES (AT3G01560), which also form large condensates reminiscent of the gel-like condensates we observed for the ΔDS FLOE1 mutant (FIG. 4D). Broadening our search, we found FLOE homologs in all plant lineages, even in ones preceding seed evolution (FIG. 4E-F, FIGS. 12-13). Phylogenetic analysis revealed the emergence of two major Glades (FLOE1-like and FLOE2-like), which show conserved variation in their disordered domains (FIG. 4G). By testing FLOE homologs across the plant kingdom, we have provided evidence for phenotypic variation in phase separation that mirrors our engineered FLOE1 mutants (FIG. 4H, FIGS. 12-13), highlighting the potential for such functional variation being used as a substrate for natural selection to act on.

Phase separation is emerging as a universal mechanism to explain how cells compartmentalize biomolecules. Recent work in yeast suggests that phase separation of prion-like and related proteins is important for their function (22, 23), but this picture is less clear for multicellular organisms, especially since aggregation of these proteins is implicated in human disease (24). There is evidence suggesting the functionality of prion-like condensates in plants (25-27) and flies (28), but strong in vivo evidence for a functional role of the emergent properties of phase separation remains lacking. While conformational switches between liquid and solid-like states of yeast prions can drive functional phenotypic variability via bet-hedging strategies (13, 23), we provide evidence that the same is true for a multicellular organism. Plant seed germination follows a bet-hedging strategy by spreading the risk of potential deleterious conditions (e.g., drought) across different phenotypes in a population (19-21). Our data show that altering both FLOE1 expression levels and its material properties can tune these strategies in different environments. While the exact molecular mode of action of this newly discovered protein is still unclear, RNAseq analysis suggests that its function is upstream of key germination pathways in a stress-dependent manner (FIG. 14A-B). Not to be bound by theory, but one hypothesis is that FLOE1 acts as a molecular glue helping to stabilize the desiccated glassy state, and this is supported by an age-dependent loss of germination potential for floe1-1 seeds (FIG. 14C). This also indicates that the reversibility of FLOE1 condensation between the dry and the imbibed state is important for its function, which is in line with the gain-of-function phenotype we observed with the irreversible DS mutant. Even though FLOE1 is so far the only reported protein to undergo hydration-dependent phase separation, it is likely that similar processes occur in a wide variety of organisms with quiescent desiccated life stages, including human pathogens 29-31). Moreover, the large repertoire of FLOE sequence variation in the plant lineage suggests the possibility that natural populations may have used phase separation to fine-tune biological function to their ecological niches.

All references, including publications, accession numbers, patent applications, and patents, cited in the disclosure are hereby incorporated by reference for the purpose for which it is cited to the same extent as if each reference were individually and specifically indicated to be incorporated by reference.

### REFERENCES CITED IN DETAILED DESCRIPTION

1. S. Yashina et al., Regeneration of whole fertile plants from 30,000-y-old fruit tissue buried in Siberian permafrost. Proc Natl Acad Sci U S A 109, 4008-4013 (2012).

N. Sam et al., Staying Alive: Molecular Aspects of Seed Longevity. Plant Cell Physiol 57, 660-674 (2016).

3. J. Buitink, O. Leprince, Intracellular glasses and seed survival in the dry state. C R Biol 331, 788-795 (2008).

4. O. Leprince, J. Buitink, Introduction to desiccation biology: from old borders to new frontiers. Planta 242, 369-378 (2015).

5. L. Raijou of al., Seed germination and vigor. Annu Rev Plant Biol 63, 507-533 (2012).

6. B. Bai et al., Ecotypic variability in the metabolic response of seeds to diurnal hydration-dehydration cycles and its relationship to seed vigor. Plant Cell Physiol 53, 38-52 (2012).

7. I. Kramer, F. V. Minibayeva, R. P. Beckett, C. E. Seal, What is stress? Concepts, definitions and applications in seed science. New Phytol 188, 655-673 (2010).

8. M. Schmid et al., A gene expression map of Arabidopsis thaliana development. Nat Genet 37, 501-506 (2005).

9. R. S. Austin et al., New BAR tools for mining expression data and exploring Cis-elements in Arabidopsis thaliana. Plant J 88, 490-504 (2016).

10. Y. Shin, C. P. Brangwynne, Liquid phase condensation in cell physiology and disease. Science 357, (2017).

11. S. Boeynaems et al., Protein Phase Separation: A New Phase in Cell Biology. Trends Cell Biol 28, 420-435 (2018).

12. S. Alberti, R. Hoffmann, O. King, A. Kapila, S. Lindquist, A systematic survey identifies prions and illuminates sequence features of prionogenic proteins. Cell 137, 146-158 (2009).

13. R. Halfmann et al., Prions are a common mechanism for phenotypic inheritance in wild yeasts. Nature 482, 363-368 (2012).

14. E. Gutierrez-Beltran, P. N. Moschou, A. P. Smertenko, P. V. Bozhkov, Tudor staphylococcal nuclease links formation of stress granules and processing bodies with mRNA catabolism in Arabidopsis. Plant Cell 27, 926-943 (2015).

15. M. C. Munder et al., A pH-driven transition of the cytoplasm from a fluid -to a solid-like state promotes entry into dormancy. Elife 5. (2016).

16. K. A. Burke, A. M. Janke, C. L. Rhine, N. L. Fawzi, Residue-by-Residue View of In Vitro FUS Granules that Bind the C-Terminal Domain of RNA Polymerase II. Mol Cell 60, 231-241 (2015).

17. J. Wang et al., A Molecular Grammar Governing the Driving Forces for Phase Separation of Prion-like RNA Binding Proteins. Cell 174, 688-699 e616 (2018).

18. E. W. Martin et al., Valence and patterning of aromatic residues deterniine the phase behavior of prion-like domains. Science 367, 694-699 (2020).

19. J. R. Greiner, D. L. Venable, Bet hedging in desert winter annual plants: optimal germination strategies in a variable environment. Ecol Lett 17, 380-387 (2014).

20. G. Johnston, G. W. Bassel, Identification of a bet-hedgina network motif generating noise in hormone concentrations and germination propensityin Arabidopsis. J R Soc Interface 15, (2018).

21. P. Villa Martin, M. A. Munoz, S. Pigolotti, Bet-hedgina strategies in expanding populations. PLoS Comput Biol 15, e1006529 (2019).

22. J. A. Riback et al., Stress-Triggered Phase Separation Is an Adaptive, Evolutionarily Tuned Response. Cell 168, 1028-1040 e1019 (2017).

23. T. M. Franzmann et al., Phase separation of a yeast priori protein promotes cellular fitness. Science 359. (2018).

24. O. D. King, A. D. Gitler, J. Shorter, The tip of the iceberg: RNA-binding proteins with prion-like domains in neurodegenerative disease. Brain Res 1462. 61-80 (2012).

25 S. K. Powers et al., Nucleo-cytoplasmic Partitioning of ARF Proteins Controls Auxin Responses in Arabidopsis thaliana. Mol Cell 76, 177-190 e175 (2019).

26. S. Chakrabottee et al., Luminidependens (LD) is an Arabidopsis protein with prion behavior. Proc Natl Acad Sci USA 113, 6065-6070 (2016).

27. X. Fang et al., Arabidopsis FLL2 promotes liquid-liquid phase separation of polyadenylation complexes. Nature 569, 265-269 (2019).

28. B. Bakthavachalu et al., RNP-Granule Assembly via Ataxin-2 Disordered Domains Is Required for Long-Tenn Memory and Neurodegeneration. Neuron 98, 754-766 e754 (2018).

29. T. C. Boothby, G. J. Pielak, Intrinsically Disordered Proteins and Desiccation Tolerance: Elucidating Functional and Mechanistic Underpinnings of Anhydrobiosis. Bioessays 39, (2017).

30. J. Esbelin, T. Santos, M. Hebraud, Desiccation: An environmental and food industry stress that bacteria commonly face. Food Microbiol 69, 82-88 (2018).

31. V. Giarola, Q. Hou, D. Bartels, Angiosperm Plant Desiccation Tolerance: Hints from Transcriptomics and Genome Sequencing. Trends Plant Sci 22, 705-717 (2017).

### MATRIALS AND METHODS FOR EXAMPLES

### Identification and Analysis of the Seed Proteome

Arabidopsis thaliana genes were scored via the Expression Angler tool based on similarity to a “Developmental Map” expression pattern with “High Relative Expression” in “Dry Seed” and “Low Relative Expression” for all other tissues (http address bar.utoronto.ca/ExpressionAngler/) (I). The output were then normalized to Z-scores (data not shown) and genes were considered as seed-specifie if they had a Z score of 3 or higher. The MobiDB-lite disorder scores of each gene in the “Z>3” and “Z<3” groups were retrieved from the MobiDB (version 3.1) A. thailana dataset (http address mobidb.bionnipd.itldataset) (2), and their amino acid profiles were obtained using the protr package (3) in R. Genes in the “Z>3” group were then checked for the presence of a predicted prion-like domain (4). For FLOE1 disorder prediction we used PONDR VSL2 (web address pondr.com) (5) and for identifying its prion-like domain we used PLAAC (web address.wi.mit.edu/) (6).

### Plant Growth Conditions

Arabidopsis thaliana plants from which seeds were harvested for the experimental assays were grown in soil (PRO-MIX® HP Mycorrhizae) inside growth cabinets (Percival) held at 22° C. and 55% humidity with a 16/8 hour photoperiod (32-watt T8 light bulbs emitting 3000k white light). Seeds were stratified for 3 days at 4° C. in the darkness to break dormancy. Plants from each line were randomly distributed and rotated every day until bolting to minimize environmental variations. When siliques began to mature, humidity was decreased to 45% as recommended by the Arabidopsis Biological Resource Center (see, ftp://ftp.arabidopsis.org/ABRC/abrc_plant_growth.pdf). Harvested seeds were air-dried for a week before being stored in Eppendorf tubes at 4° C.

Arabidopsis thaliana plants that were used for line propagation were grown in soil (PRO-MIX® HP Mycorrhizae) inside chambers held at 22° C. with a 16/8 hour photoperiod. Seeds were stratified fbr 3 days at 4° C. in the darkness to break dormancy.

Nicotiana benthamiana plants were grown in soil (PRO-MIX® PDX) inside chambers held at 22° .C with a 16/8 hour photoperiod.

### Plant Material

floe1-1 T-DNA mutant:

The mutant line floe1-1 (SALK_048257C) was obtained from the Arabidopsis Biological Resource Center (ARRC') and gcnotyped using primers priFLOE1cds-FWD/REV and the Salk genotyping primer LBb1.3 (sequences not shown). It was confirmed to be a knockout mutant by RT-qPCR (FIG. 10D) as described in the RT-qPCR analyses section.

### Transgenic Lines:

Transgenic plants were generated by Agrobacterium-mediated (GV3101 strain) transformation (7) of floe1-1 with the constructs described in the Plant plasmid construction section, with the exception of the control transgenic line overexpressing YFP-FLAG used in FIG. 7A that was generated by introducing the transgene into Col-0. Transgenic seedlings (T₁) were selected with Basta and T₂ lines containing only one T-DNA construct were selected for further characterization by determining the Mendelian segregation ratio (3:1) of Basta-resistant seedlings in their progeny. Homozygote T₂ lines were then identified by verifying that T₃ seedlings (their progeny) were all Basta-resistant.

### CRISPR Lines:

FLOE1 CRISPR lines were generate (using the Staphylococcus aureus CRISPR-Cas9 system (8) and by following the protocol described in (web address botanik.kit.edu/molbio/940.php). A region within the QPS-rich region was identified as having a NNGGGT protospacer adjacent motif (PAM) downstream of a protospacer sequence (5′TTACAGCCCCCAGACTGGC3′) that did not have any significant similarities to other genomic regions. The corresponding guide RNA was inserted in the Bbsil site of the pEn-Sa-Chimera vector through digestion-ligation following hybridization of the oligo duplex priCRISPR-FWD/REV. The resulting sgRNA coding vector was then transferred to pDe-Sa-CAS9 through LR recombination, The final binary destination vector was then used to transform Agrobacterium (GV3101 strain), which was used to transform Col-0 plants using the floral dip method (7). Seeds obtained from the T₀ parental lines were sown on MS media (0.5X Murashige and Skoog basal salt mixture (MS) media (PhytoTechnologies Laboratories) (pH 5.7), supplemented with 0.8% agar (Difco) and 1% sucrose (Sigma-Aldrich)) supplemented with 30 mg/L Kanamycin (G-Biosciences) for selection of successfully transformed transgenics, Selected T₁ seedlings were then transferred to soil to mature. Genomic DNA was extracted from mature rosette leaves of each of these T₁ plants and the Cas9-recognition site within FLOE1 was amplified through PCR with Phusion DNA polymerase (Thermo Fisher Scientific) using primers prigenoCRISPR-FWD/REV. Sequencing (Sequetech Inc.) of the amplicons revealed that 12 plants demonstrated heterogenous sequences at the targeted region, which were subsequently selected for growing the T₂ generation. For each selected T₁ plant, 8 T₂ progeny were grown, and PCR amplification followed by sequencing of the FLOE1 amplicon was again performed on genomic DNA extracted from mature rosette leaves. Four individuals from this T₂ generation (floe1-2, floe1-3, floe1-4, floe1-5) presented different homozygous mutations in the FLOE1 amplicon, leading to frameshift mutations and pre-mature stop codons in the QPS region, and were selected for further assays.

### Plant Plasmid Construction

Constructs were generated using the Gateway system Titrogen the pGWB601-661 collection (9) as follows:

Transgenes for Arabidopsis experiments: FLOE1's genomic region spanning its promoter, as predicted by AGRIS (10), to its last coding codon was amplified by PCR from Col-0 DNA (extracted with IDNeasy Plant Mini Kit (Qiagen)) using the prigFLOE1-FWD/REV primers. The amplicon was first cloned into pDONR221 (Thermo Fisher Scientific) using BP Clonase II (Thermo Fisher Scientific) and then subcloned into pGWB604, pGWB610 and pGWB633 using LR Clonase II (Thermo Fisher Scientific) to generate pFLOE1p:FLOE1-GFP, pFLOE 1p:FLOE1-FLAG and pFLOE1p:FLOE1-GUS respectively.

FLOE1p:FLOE1ΔDS-GFP, FLOE 1p:FLOE1ΔQPS-GFP, and FLOE1p:FLOE1ΔDUF-GFP were obtained by moditing pFLOE1p:FLOE1-GFP using the Q5 Site-Directed Mutagenesis Kit (New England Biolabs) with primers priDSdeletion-FWD/REV, priQPSdeletion-FWD/REV, and priQPSdeletion-FWD/REV, and priDUFdeletion-FWD/REV respectively.

An entry vector containing the YFP gene was donated by Dr. Zhiyong Wang (Carnegie Institution for Science, USA) and another one, G18395, containing FLOE1's coding sequence was obtained from ABRC. The two genes were then transferred from the entry vector into the binary vector pB7HFC3_0 (11) using Gateway cloning (Life Technologies), to create the vector p35S:YIT-FLAG and p35S:FLOE1-FLAG.

Transgenes for tobacco (Nicotiana benthamiana) experiments:

A. Arabidopsis genes: The coding sequences of FLOE1's isoforms, FLOE1.1 and FLOE1.2 were amplified by PCR from the entry vector G18395 using priFLOE1.1-FWD/REV and priFLOE1.2-FWD/REV and then BP recombined into pDONR221 (Thermo Fisher Scientific). These were then transferred by LR recombination into pGWB605 to generate p35S:FLOE1.1-GFP and p35S:FLOE1.2-GFP. Similarly, p35S:FLOE1.2-RFP was generated by subcloning FLOE1.2 into pGWB660. The N-terrninal version p35S:GFP-FLOE was generated by LR recombination of G18395 into pGWB606. To generate p35S:FLOE2-GFP and p35S:FLOE3-GFP, the coding sequences of FLOE2 and FLOE3 were obtained from 5-day old Col-0 seedlings cDNA by PCR amplification using Phusion DNA polymerase (Thermo Fisher Scientific) and the primers priFLOE2 -FWD/REV and priFLOE3-FWD/REV. Total cDNA was obtained by reverse transcription using M-MLV Reverse Transcriptase (Thermo Fisher Scientific) from total RNA extracted with the RNeasy Plant Mini Kit (Qiagen). The FLOE2 and FLOE3 amplicons were then BP recombined into pDONR221 before being transferred into pGWB605 by LR recombination.

B. Mutated FLOE1 versions: FLOE1 wt, FLOE1Δnucl, FLOE1ΔCC, FLOE1ΔQPS, and FLOE1-QPS-15×Y-S were amplified from the corresponding human expression vectors described in Human plasmid construction using prihFLOE1-FWD/REV and BP recombined into pDONR221 (Thermo Fisher Scientific) before being transferred by LR recombination into pGWB605 to generate p35S:wt.FLOE1-GFP, p35S:FLOE1Δnucl-GFP, p35S:FLOE1ΔCC-GFP, p35S:FLOE1ΔQPS-GFP, and p35S:FLOE1-QPS-15×Y-S-GFP. p35S:FLOE1ΔDS-GFP and p35S:FLOE1ΔDUF-GFP were obtained by the same process but with different primer pairs: prihFLOE1ΔDS-FWD/prihFLOE1-REV and prihFLOE1-FWD/prihFLOE1ΔDUF-REV, respectively.

C. Non-Arabidopsis FLOE1 homologs: Protein sequences for all FLOE1 homologs shown in FIG. 4 were obtained from UniProt (12) and Phytozome v12.1.5 (13). Their corresponding DNA sequences were generated with codon-optimization for Nicotiana benthamiana expression using IDT's codon optimization tool (web address idtdna.com/CodonOpt) The sequences were synthesized by GenScript Biotech Corporation (Piscataway, NJ) with flanking attB sites for subsequent BP cloning into pDONR221 (Thermo Fisher Scientific). They were then subcloned into pGWB605 by LR recombination to generate p35S:HOMOLOG-GFP constructs (where HOMOLOG refers to the relevant FLOE1 homolog).

### FLOE Homologs Analysis

Phylogenetic tree construction: All Viridiplantae protein sequences containing the highly-conserved DUF1421 domain were retrieved from UniProt (12). After removal of duplicates due to re-annotations, the remaining 791 sequences were submitted to the phylogenetic analysis tool, NGPhylogeny,fr (14) with default settings. The FastIVIE Output Tree was then uploaded to iTOL (version 5) (15) for tree visualization.

QPS and DS domains lengths: All monocot and eudicot sequences from the FLOE1 and FLOE2/3 groups were aligned using the msa package (version 1.20,0) in R (16). The DS and QPS reions of the homologs were defined as aligning to the DS and QPS regions of FLOE1. The lengths of these regions were used for subsequent analysis.

Alignments: The figure showing the alignment and protein characteristic of select FLOE1 homologs was conducted using the msaPrettyPrint( )function of the msa package (16) in R and MacTex.

### Tobacco Infiltration

Agrobacterium cultures (GV3101 strain) carrying the relevant constructs were grown overnight at 28° C., in LB broth (Fisher BioReagents) containing 25 mg/L rifampicin (Fisher BioReagents), 50 mg/mL gentamicin (GoldBio) and 50 mg/L spectinomycin (GoldBio). Cultures were washed four times with infiltration buffer (10 mM MgCl₂ (omniPur, EMD), 10 mM MES (pH 5.6) (J. T. Baker) and 100 uM acetosyringone (Sigma-Aldrich)) and diluted to reach an OD₆₀₀ of 0.8. Fully expanded 3^{rd}, 4^{th} or 5^{th} leaves from 6-week-old tobacco plants were infiltrated with these diluted Agrobacterium cultures using Monoject 1 mL Tuberculin Syringes (Covidien). For the FLOE1,1-GFP and FLOE1,2-RFP colocalization experiment, an equal amount of each culture was pre-mixed before infiltration. For each construct or combination of constructs, at least three individual tobacco plants were infiltrated.

### Germination Experiments

Seeds were first sterilized by vortexing in 70% ethanol for 5 minutes after which the solution was removed and replaced with 100% ethanol. Seeds were then placed on pre-sterilized filter papers (Grade 410, VWR) and left to dr in a laminar flow hood. Sterilized seeds were then sown on square petri dishes (120×120 wide×15 mm high (VWR)) containing 40 mL of MS media (0.5X Murashige and Skoog basal salt mixture (MS) media (PhytoTechnologies Laboratories) (pH 5.7), supplemented with 0.8% agar (Difco) and 1% sucrose (Sigma-Aldrich)) supplemented with NaCl (Sigma-Aldrich) and mannitol (Sigma-Aldrich) at the concentrations indicated in the manuscript. Plates were then sealed with micropore surgical tape (3M) and covered in aluminum foil befbre being placed at 4° C. After exactly 120 h (5 days) of stratification to break seed dormancy, plates were transferred to a 24 h light (17-watt T8 light bulbs emitting 4100k white light), 22° C. growth cabinet (Percival). Germination (identified by radicle protrusion) was counted under a dissecting microscope the following day for the normal conditions and 15 days later for the stress conditions.

Germination experiments were performed on seeds from three independent batches of plants (A. B, and C) grown as described in the Plant growth conditions section.

Batch A (FIG. 3, FIG. 10E-H, FIG. 11): Forty Col-0 and floe1-1 plants were grown alongside ten plants of each of the following lines were: four independent CRISPR lines (floe1-2, floe1-3, floe1-4, floe1-5), five independent pFLOE1p:FLOE1-GFP lines, two independent pFLOE1p:FLOE1-FLAG lines, one pFLOE1p:FLOE1-GUS line, three independent FLOE1p:FLOE1ΔDS-GFP lines, four independent FLOE1p:FLOE1ΔQPS-GFP lines, and three independent FLOE1p:FLOE1ΔDUF-GFP lines. For each line, seeds from five plants were randomly pooled together which resulted in two biological replicates of each CRISPR and complemented line, and eight biological replicates of Col-0 and floe1-1. For each biological replicate and each germination condition (0, 80 mM, 100 mM, 120 mM, 140 mM, 160 mM, 180 mM, 195 mM, 200 mM, 210 mM, 220 mM, 230 mM and 240 mM NaCl), three technical replicates were conducted. At the end of the 230 mM NaCl germination experiment (day 15), the seeds that did not germinate were rinsed in sterile double distilled water and sown on normal MS media. Two days later, germination was scored to test whether they maintained their germination potential.

Batch B (FIG. 10A-D): Fourteen Col-0 and twenty-seven floe)-1 plants were grown alongside siz plants of each of the following lines: three independent pFLOE1p:FLOE1-GFP lines, two independent pFLOE1p:FLOE1-FLAG lines, one pFLOE1p:FLOE1-GUS line, and two independent 35S:FLOE1-FLAG lines. The 35S:FLOE1-FLAG lines failed to express FLOE1 as revealed by RT-qPCR (FIG. 10D) and were therefore chosen as transgenic controls. Seeds from each individual plant were sown on media supplemented with either mannitol (400 mM) or NaCl (190 mM, 205 mM and 220 mM). For each biological replicate and each germination condition, three technical replicates were conducted.

Batch C (FIG. 14C): 5 floe1-1 plants and 5 Col-0 plants were alternated within the same flat. Seeds from each individual plant were harvested and aged in Eppendorf tubes placed inside an opaque box stored at room temperature for 42 months (3,5 years). They were then sown on MS medium (See Plant growth conditions section). For each biological replicate, three technical replicates were conducted.

### Embryo Dissection and Assays:

Salt, inainiitol, sorbitol, cycloheximide and water assays: Seeds of the relevant GFP-tagged lines were submerged in either glycerin or in solutions of NaCl (Sigma-Aldrich). mannitol (Sigma-Aldrich), sorbitol (Sigma-Aldrich), cycloheximide (GoldBio) or double distilled water at concentrations indicated in the manuscript for 15-30 min (NaCl: 0, 0.2M, 0.4M, 0.6M, 0.8M, 1M, 1.2M, 1.4M, 1.6M, 1.8M, 2M; mannitol: 0, 950 mM; sorbitol: 0, 0.725M, 1.45M; cycloheximide: 1 g/L). They were then dissected to remove the seed coat and imaged by confocal microscopy (see Plant microscopy and image analysis). As controls, 35S: (11) and Col-0 seeds were dissected in water to verify that. GFP alone could not induce condensate formation and to indicate the level of autofluorescence of the protein storage vacuoles in the absence of GFP, respectively.

Condensate reversibility assays: Three different types of FLOE1 condensate reversibility assays were performed: 1) Embryos from dry seeds were first dissected in glycerin as described above, and after imaging, glycerin was washed off from the embryos with water and the same embryos were imaged in water; 2) Seeds were submerged in water for 1 hr before being transferred to 2M NaCl for 10 min and imaged and vice versa (1 h in 2M NaCl followed by 10 min in water); and 3) Seeds were submerged in water overnight and then left to dry for an additional day. Seeds were then either dissected in glycerin to obtain the condensate state of the dry seeds or in water to assess the ability to re-form condensates.

End of germination experiment analysis: At the end of the 230 mM NaCl germination experiment described in the Germination Experiments section (15 days in light following 5 days of stratification on MS media supplemented with 230 mM NaCl), seeds that did not germinate were either: 1) dissected directly in glycerin to maintain the hydration state of the seed; or 2) transferred first to normal MS media and dissected in glycerin two hours later. Dissected embryos were then imaged by confocal microscopy to obtain a snapshot of their final condensate state (see Plant microscopy and image analysis).

Developmental stages: FLOE1p:FLOE1-GFP and 35S:YFP-FLAG flower buds were self-crossed 11, 8, 6 and 4 days before dissection to obtain developing siliques carrying embryos at mature, torpedo, heart and globular stages respectively. Seeds from the various developmental stages were dissected either in glycerin or water and imaged by confocal microscopy (see Plant microscopy and image analysis).

### GUS Staining

FLOE1p:FLOE1-GUS seeds carrying embryos at different stages of maturation were incubated at 37° C. overnight in GUS staining solution (17)In the case of dry seeds, seed coats were first removed as they were impermeable to the staining solution and incubated at 37° C. for one hour in GUS staining solution. Following the incubation, samples were destained in 70% ethanol at room temperature for 24 hours and embryos were dissected out (in the case of developing siliques) before imaging. Pictures were taken with a compound microscope (Nikon) and dissecting scope (Leica MZ6 microscope).

### Plant Microscopy and Image Analysis

Image acquisition: Embryos and tobacco leaves were imaged at room temperature on a LECIA TCS SP8 laser scanning confocal microscope in resonant scanning mode using the LASX software. All samples were imaged with a Hf PL APO CS2 63X/1.20 water objective with the exception of embryos submerged in glycerin that were imaged with a 63X/1.30 glycerin objective and of embryos of early developmental stages that were imaged with a HC PL APO CS2 20×/0.75 dry objective. GFP, RFP, and YFP fluorescence was detected by exciting with a white light laser at 488 nm, 561 nm and 514 nm, respectively, and by collecting emission from 500-500 nm, 591-637 nm and 524-574 nm, respectively, on a HyD SMD hybrid detector (Leica) with a lifetime gate filter of 1-6 ns to reduce background autofluorescence due to chlorophyll (tobacco) or protein storage vacuoles (embryos). Z-stacks were collected with a bidirectional 96-line averaging while single-frame images (tobacco images displayed in the publication) were collected with a bidirectional 1024-line averaging. For the colocalization experiments, samples were imaged sequentially between each line to ensure that the colocalization signals were not due to bleed-throughs. Images displayed in the publication were representative of at least three biological replicates for each construct (tobacco) or line (Arabidopsis). All samples that were compared in the publication were imaged with the same magnification and laser intensity.

Heterogeneity analysis: For each radicle and experimental condition, maximum projection images of their corresponding Z-stacks were obtained using the LASX software. ROIs were then manually drawn around each individual cell to obtain their standard deviation (RMS) and mean intensity levels. Heterogeneity scores were obtained by dividing the standard deviation by the mean. Between 363 and 461 cells were measured per embryo with a total of 3 embryos per condition. Cells were characterized as exhibiting FLOE1 condensates if their heterogeneity score was higher than the top 5 percentile of the 2M NaCl condition (heterogeneity cut-off=0.3 a.u.).

Granule size: Individual slices of a radicle Z-stack were analyzed using FIJI (18). Individual granules were identified using a threshold, followed by a watershed, and subsequently measured for their area. A total of 3-4 embryos per condition were analyzed.

### Seed Phenotyping

Seed weight: Twelve and fourteen biological replicates of floe1-1 and Col-0 seeds, respectively, were used for the seed weight analysis. Seeds were weighed on a Sartorius M2P scale in batches of nine to twenty seeds and the process was replicated three times per biological replicate. The average weight per seed was calculated and used for subsequent statistical analysis.

Seed size and aspect ratio: Fourteen and sixteen biological replicates of floe1-1 and Col-0 seeds, respectively, were used for the seed size and aspect ratio analysis. Seed images were scanned using a Canon CanoScan LiDE 700 F (Canon Inc). All images were scanned at 600 dpi and, for ease of collection, the seeds were placed in transparent bags before scanning. The number of seeds per image varied, but ten seeds per sample were randomly selected and analyzed for area quantification and aspect ratio using ImageJ (version 2.0.0) (19). This process was replicated ten times per biological replicate to obtain a total of hundred seeds per biological replicate.

### RNA Extraction From Seeds

DNA-free total RNA was extracted from seeds and siliques (20). The extraction buffer utilized 0.5% β-mercaptoethanol. RNA quantity and purity from all samples were assessed using a NanoDrop Spectrophotometer (Thermo Fisher Scientific).

### RT-qPCR Analyses

cDNA was synthesized from I pg of extracted RNA using M-MLV Reverse Transcriptase (Invitrogen), per manufacturer's protocol. qPCR was performed using the SensiFAST SYBR No-ROX Kit (Bioline). Primers used to quantify FLOE1 expression were priqPCRFLOE1set1-FWD-REV, with the exception of the qPCRs conducted on the CRISPR lines as well as on siliques and seeds from different developmental stages (FIG. 6B) where priqPCRFLOE1set2-FWD/REV were used. The reference gene that was used to normalize, At5G25760 (PEX4), was chosen for consistent expression in seeds as reported before (21). The corresponding primer pair, priAT5G25760-FWD/REV, was the one reported in reference (22). Reactions were run on 96-well plates in the LightCycler® 480 Instrument II system and were repeated three times.

### RNA-sets Experimental Conditions and Analysis

Experimental design: Six conditions were utilized in the RNA-seq analysis: 1) dry floe1-1 seeds; 2) dry Col-0 seeds; 3) imbibed floe1-1 seeds; 4) imbibed col-0 seeds; 5) salt-stressed imbibed floe1-1 seeds; and 6) salt-stressed imbibed Col-0 seeds. Three biological replicates corresponding to pooled seeds from 20 different plants were performed per condition, with 50 mg of mature seeds used per biological replicate. For conditions (1) and (2), RNA was extracted directly from dry seeds using the protocol described in the RNA extraction from seeds section. For conditions (3) and (4), and for each biological replicate, dry seeds were sown onto separate but identical agar plates of normal MS media conditions (0.5X Murashige and Skoog basal salt mixture (MS) media (PhytoTechnologies Laboratories) (pH 5.7), supplemented with 0.8% agar (Difco) and 1% sucrose (Sigma-Aldrich)) and cold-stratified for 5 days at 4° C. in the dark. All plates were subsequently transferred to and held in a growth cabinet (Percival) for exactly 4 hours under light and 22° C. After the 4-hour incubation, imbibed seeds were scraped from each plate and transferred to a clean mortar and pestle and ground in liquid nitrogen. Conditions (5) and (6) were conducted in parallel and using the exact same experimental setting with the only difference being that the MS media was supplemented with 220 mM NaCl.

For all biological replicates, 2 μL of extracted RNA was combined with 2 μL of DNase/RNase-free dH₂O for a 1:2 dilution and sent to the Stanford University Protein and Nucleic Acid Facility for quantification and quality analysis using an Agilent 2100 Bioanalyzer. After analysis, 5 μL of extracted RNA was combined with 20 μL of DNase/RNase-free dH₂O for a 1:5 dilution and sent to Novogene Corporation Inc. (Sacramento, CA) for RNA-seq library preparation (250-300 by insert cDNA library) and sequencing (2×150 by paired-end reads on an Illumina Platform).

Analysis: Reads were mapped with HISAT2 to the Arabidopsis thaliana TAIR10 reference genome using the Galaxy (Version 2.1.0+galaxy5) web platform (https usegalaxy.eu) (23). The resulting BAM files were then analyzed on R using the DESeq2 (24) and T×DB.Athaliana.BioMart.plantsmart28 (Bioconductor) packages. Genes with padj<0.05 were considered differentially expressed. Gene ontology and KEGG enrichment of the differentially expressed genes was obtained using g:Profiler (biit.cs.utee/gprofiler/gost) (25).

### Human Plasmid Construction

FLOE1 and derived mutant constructs for expression in human cells were optimized for human expression and generated through custom synthesis and subcloning into the pcDNA3.1+N-eGFP backbone by Genscript (Piscataway, USA).

### Human Cell Culture and Microscopy

U2OS cells (ATCC, HTB-96) were grown at 37° C. in a humidified atmosphere with 5% CO₂ for 24 h in DMEM, high glucose, GlutaMAX+10% FBS and pen/strep (Thermo Scientific). Cells were transiently transfected using Lipofectamine 3000 (Invitrogen) according to manufacturer's instructions. Cells grown on cover slips were fixed 24 h after transfection in 4% formaldehyde in PBS. Slides were mounted using ProLong Gold antifade reagent (Life Technologies). Confocal images were obtained using a Zeiss LSM 710 confocal microscope. Images were processed using FIJI (18).

### FRAP Measurements in Human Cells

U2OS cells were cultured in glass bottom dishes (Ibidi) and transfected with GFP-FLOE1 constructs as described above. After 24 hr GFP-FLOE1 condensates were bleached and fluorescence recovery after bleaching was monitored using Zen software on a Zeiss LSM 710 confocal microscope with incubation chamber at 37° C. and 5% CO₂. Data were analysed as described previously (28). In brief, raw data were background subtracted and normalized using Excel, and plotted using GraphPad Prism 8.4.1 software.

### Statistical Analysis.

All data was analyzed using Graphpad Prism 8.4.1 and Excel. Statistical tests details are shown in the figure legends.

### REFERENCES CITED IN MATERIALS & METHODS

1. R. S. Austin et al., New BAR tools for mining expression data and exploring Cis-elements in Arabidopsis thaliana. Plant J 88, 490-504 (2016).

2. D. Piovesan al., MobiDB 3.0: more annotations for intrinsic disorder, confortnational diversity and interactions in proteins. Nucleic Acids Res 46, D471-D476 (2018).

3. N. Xiao, D. S. Cao, M. F. Zhu, Q. S. Xu, protr/ProtrWeb: R package and web server for generating various numerical representation schemes of protein sequences. Bioinformatics 31, 1857-1859 (2015).

4. S. Chakrabortee et al., Luminidependens (LD) is an Arabidopsis protein with prion behavior, Proc Natl Acad Sci USA 113, 6065-6070 (2016).

B. Yue, R. L. Dunbrack, R. W. Williams, A. K. Dunker, V. N. Uversky, PONDR-FIT: a meta-predictor of intrinsically disordered amino acids. Biochim Biophys Acta 1804, 996-1010 (2010).

6. A. K. Lancaster, A. Nutter-Upham, S. Lindquist, O. D. King, PLAAC: a web and command-line application to identify proteins with prion-like amino acid composition. Bioinformatics 30, 2501-2502 (2014).

7. S. J. Clough, Floral dip: agrobacterium-mediated germ line transformation. Methods Mol Biol 286, 91-102 (2005).

8. J. Steinert, S. Schiml, F. Fauser, H. Puchta, Highly efficient heritable plant genome engineering using Cas9 orthologues from Streptococcus thermophilus and Staphylococcus aureus. Plant J 84, 1295-1305 (2015).

9. T. Nakagawa et al., Development of series of gateway binary vectors, pGWBs, for realizing efficient construction of fusion genes for plant transformation. J Biosci Bioeng 104, 34-41 (2007).

10. R. V. Davuluri et al,, AGRIS: Arabidopsis gene regulatory information server, an information resource of Arabidopsis cis-regulatory elements and transcription factors. BMC Bioinformatics 4, 25 (2003).

11. F. Bossi et al., Systematic discovery of novel eukaryotic transcriptional regulators using sequence homology independent prediction. BMC Genomics 18, 480 (2017).

12. C. UniProt, UniProt: a worldwide huh of protein knowledge. Nucleic Acids Res 47, D506-D515 (2019).

13. D. M. Goodstein et al., Phytozome: a comparative platform for green plant genomics. Nucleic Acids Res 40, D1178-1186 (2012).

14. E Lemoine et al., NGPhylogeny.fr: new generation phylogenetic services for non-specialists. Nucleic Acids Res 47 W260-W265 (2019).

I. Letunic, P. Bork, Interactive Tree Of Life (iTOL) v4: recent updates and new developments. Nucleic Acids Res 47, W256-W259 (2019).

16. U. Bodenhofer, E. Bonatesta, C. Horejs-Kainrath, S. Hochreiter, msa: an R package for multiple sequence alignment. Bioinformatics 31 3997-3999 (2015).

17. R. A. Jefferson, T. A. Kavanagh, M. W. Bevan, GUS fusions: beta-glucuronidase as a sensitive and versatile gene fusion marker in higher plants. EMBO J 6. 3901-3907 (1987).

18. J. Schindelin et al., Fiji: an open-source platfortn for biological-image analysis. Nat Methods 9, 676-682 (2012).

19. C. A. Schneider, W. S. Rasband, K. W. Eliceiri, NIH Image to ImageJ: 25 years of image analysis. Nat Methods 9, 671-675 (2012).

20. L. Meng, L. Feldman. A rapid TRIzol-based two-step method for DNA-free RNA extraction from Arabidopsis siliques and dry seeds. Biotechnol J 5, 183-186 (2010).

21. B. J. Dekkers et al., Identification of reference genes for RT-qPCR expression analysis in Arabidopsis and tomato seeds. Plant Cell Physiol 53, 28-37 (2012).

22. T. Czechowski, M. Stitt, T. Altmann, M. K. Udvardi, W. R. Scheible, Genome-wide identification and testing of superior reference genes for transcript normalization in Arabidopsis. Plant Physiol 139, 5-17 (2005).

23. E. Afgan et al., The Galaxy platform for accessible, reproducible and collaborative biomedical analyses: 2018 update. Nucleic Acids Res 46, W537-W544 (2018).

24. M. I. Love, W. Huber, S. Anders, Moderated estimation of fold change and dispersion for RNA-seq data with DESeq2. Genome Biol 15, 550 (2014).

25. U. Raudvere et al., g:Profiler: a web server for functional enrichment analysis and conversions of gene lists (2019 update). Nucleic Acids Res 47, W191-W198 (2019).

26. S. Nallamsetty, B. P. Austin, K. J. Penrose, D. S. Waugh, Gateway vectors for the production of combinatorially-tagged His6-MBP fusion proteins in the cytoplasm and periplasm of Escherichia coli. Protein Sci 14, 2964-2971 (2005).

27. K. A. Burke, A. M. Janke, C. L. Rhine, N. L. Fawzi, Residue-by-Residue View of In Vitro FUS Granules that Bind the C-Terminal Domain of RNA Polymerase II. Mol Cell 60, 231-241 (2015).

28. S. Boeynaerns, M. De Decker, P. Tompa, L. Van Den Bosch, Arginine-rich Peptides Can Actively Mediate Liquid-liquid Phase Separation. Bio-protocol 7, (2017).

29. K. L. McDonald, R. I. Webb, Freeze substitution in 3 hours or less. J Microsc 243, 227-233 (2011).

30. C. J. Peddie et al., Correlative and integrated light and electron microscopy of in-resin GFP fluorescence, used to localise diacylglycerol in mammalian cells. Ultramicroscopy 143, 3-14 (2014).

31. J. Waese et al., ePlant: Visualizing and Exploring Multiple Levels of Data for Hypothesis Generation in Plant Biology. Plant Cell 29, 1806-1821 (2017).

1. A method of modulating seed germination, and/or increasing seed viability the method comprising modulating FLOE1 levels in seeds of a plant compared to levels in the wildtype plant.

2. The method of claim 1, comprising decreasing endogenous FLOE1 levels in seeds, thereby enhancing germination.

3. The method of claim 1, comprising increasing FLOE1 levels in seeds, thereby decreasing germination

4. The method of claim 3, wherein increasing FLOE1 levels comprises increasing the level of endogenous FLOE1 in plant seeds.

5. The method of claim 1, the method comprising expressing a FLOE1 protein in which a DS domain or QPS domain is deleted.

6. The method of claim 1 comprising increasing FLOE1 levels in seeds of a plant, compared to FLOE1 levels in a wildtype control plant, thereby increasing seed viability.

7. A plant genetically modified by the method of claim 4 to increase levels of FLOE1 in the seeds compared to the wildtype plant.

8. A plant genetically modified by the method of claim 2 to decrease levels of endogenous FLOE1 in the seeds compared to the wildtype plant.

9. A plant comprising seeds that express a FLOE1 protein in which a DS domain or QPS domain is deleted, in which the QPS domain comprises substitutions at multiple tyrosine positions, optionally, wherein serine residues or phenylalanine residues are substituted for tyrosine residues; or in which the DS domain comprises substitutions at multiple aspartic acid residues, optionally wherein asparagine residues are substituted.

10. Seeds of a plant of claim 9.
